(19) United States
(12) Patent Application Publication
(10) Pub. No.: US 2003/0186329 A1
(43) Pub. Date: Oct. 2, 2003

(54) Use of substrate subtraction libraries to distinguish enzyme specificities

(75) Inventors: Edwin L. Madison, Del Mar, CA (US); Song-Hua Ke, San Diego, CA (US)

Correspondence Address: THE SCRIPPS RESEARCH INSTITUTE, OFFICE OF PATENT COUNSEL, TPC-8, 10550 NORTH TORREY PINES ROAD, LA JOLLA, CA, 92037, US

(73) Assignee: The Scripps Research Institute, La Jolla, CA

(21) Appl. No.: 10/348,232

(22) Filed: Jan. 21, 2003

Related U.S. Application Data
(63) Continuation of application No. 09/202,265, filed on Mar. 22, 1999, now abandoned.
Application No. 09/202,265 is a 371 of international application No. PCT/US97/09760, filed on Jan. 9, 1998.

(51) Int. Cl. G01N 33/53

(52) U.S. Cl. 435/7.1

(57) ABSTRACT

The invention provides substrate subtraction libraries and methods of using substrate subtraction libraries to identify highly selective substrates for enzymes which use peptides as substrates. The substrates identified by the present invention are useful for the construction of highly selective enzyme inhibitors.

### REFERENCE TO RELATED APPLICATION

This application claims the benefit of U.S. Provisional Application Ser. No. 60/019,495, filed Jun. 10, 1996, which is incorporated by reference, as are all references cited herein.

### GOVERNMENTAL RIGHTS

This invention was made with governmental support from the United States Government, National Institutes of Health, Grants HL52475 and HL31950; the United States Government has certain rights in the invention.

### FIELD OF THE INVENTION

The invention relates to methods for elucidating specificity differences between closely related enzymes and making enzyme inhibitors based on those differences.

### BACKGROUND OF THE INVENTION

The chymotrypsin family of serine proteases has evolved to include members with intimately related substrate specificities (Perona, J. J. & Craik, C. S., Structural basis of substrate specificity in the serine proteases, Protein Science 4: 337-360, 1995). Two of these enzymes, t-PA and u-PA, were chosen to test the hypothesis that small molecule libraries could be used to identify substrates that discriminate between closely related enzymes. These two proteases possess an extremely high degree of structural similarity (Spraggon G, Phillips C, Nowak UK, et al. The crystal structure of the catalytic domain of human urokinase-type plasminogen activator, Structure 3:681-691, 1995), share the same primary physiological substrate (plasminogen) and inhibitors (plasminogen activator inhibitor types 1 and 2), exhibit remarkably stringent substrate specificity, and play key roles in critical biological processes. Plasminogen activator inhibitors are examples of the class of molecules known as serpins (serine protease inhibitors) (Lawrence, D. A. and Ginsburg, D., in Molecular Basis of Thrombosis and Hemostasis, K. A. High, H. R. Roberts, Eds., Marcel Dekker, New York, 1995, pp. 517-543).

In general, proteases and their inhibitors, such as serine proteases and serpins, are involved in numerous biological processes in addition to fibrinolysis, such as ovulation, fertilization, embryogenesis, angiogenesis, infection and inflammation. See, generally, Katunuma, N., et al., editors, Biological Functions of Proteases and Inhibitors, Karger, Tokyo, 1994; Troll, W., and Kennedy, A. R., editors, Protease Inhibitors as Cancer Chemopreventive Agents, Plenum Press, New York, 1993; Gettins, P. G. W., et al., editors, Serpins: Structure, Function and Biology, Chapman and Hall, New York, 1996; Sandier, M., and Smith, H. J., editors, Design of Enzyme Inhibitors as Drugs, Oxford University Press, New York, 1989). One particularly important use of protease inhibitors is in the treatment of HIV infection and AIDS (Huff, J. R., and Darke, P. L., Inhibition of HIV protease: A strategy to the treatment of AIDS, in Mohan, P., and Baba, M., editors, Anti-AIDS Drug Development, Harwood Academic Publishers, Chur, 1995)

Local activation and aggregation of platelets, followed by initiation of the blood coagulation cascade (collectively part of what is referred to as the hemostatic system), assure that a fibrin clot will form rapidly in response to vascular injury (Roberts, H. R., and Tabares, A. H. (1995) in Molecular Basis of Thrombosis and Hemostasis, K. A. High, H. R. Roberts, Eds., Marcel Dekker, New York, N.Y., 1995, pp. 35-50). The presence of this clot, however, must be transient if the damaged tissue is to be remodeled and normal blood flow restored. The fibrinolytic system, which accomplishes the enzymatic degradation of fibrin, is therefore an essential component of the hemostatic system. The ultimate product of the fibrinolytic system is plasmin, a chymotrypsin family enzyme with relatively broad, trypsin-like primary specificity that is directly responsible for the efficient degradation of a fibrin clot (Castellino, F. J. (1995) in Molecular Basis of Thrombosis and Hemostasis, K. A. High, H. R. Roberts, Eds., Marcel Dekker, New York, 1995, pp. 495-515). Production of this mature proteolytic enzyme from the inactive precursor, or zymogen, plasminogen is the rate limiting step in the fibrinolytic cascade (Collen, D., and Lijnen, H. R. (1991) Blood 78, 3114-3124). Catalysis of this key regulatory reaction is tightly controlled in vivo and is mediated by two enzymes present in human plasma, u-PA and t-PA.

u-PA and t-PA are very closely related members of the chymotrypsin gene family. These two proteases possess extremely high structural similarity (Spraggon, G., et al., (1995) Structure 3: 681-691; Lamba, B., et al., (1996) J. Mol. Biol. 258: 117-135), share the same primary physiological substrate (plasminogen) and inhibitor (plasminogen activator inhibitor type 1, PAI-1) (Lawrence and Ginsburg, 1995), and, unlike plasmin, exhibit remarkably stringent substrate specificity.

In spite of their striking similarities, the physiological roles of t-PA and u-PA are distinct (Carmeliet, P.,et al. (1994) Nature 368: 419-424; Carmeliet, P., and Collen, D. (1996) Fibrinolysis 10: 195-213). Many studies (5, 6, 12-18) suggest selective inhibition of either enzyme should have beneficial therapeutic effects. Mice lacking t-PA, for example, are resistant to specific excitotoxins which cause extensive neurodegeneration in wild type mice, and mice lacking u-PA exhibit defects in the proliferation and/or migration of smooth muscle cells in a model of restenosis following vascular injury.

Either increased levels of protease inhibitors, such as PAI-1, or decreased levels may be associated with diseases. Increased levels if PAI-1 in the circulation are associated with thrombotic disease, including myocardial infarction and deep vein thrombosis, and reduced post-operative fibrinolytic activity (Lawrence and Ginsburg (1995) page 526). Conditions in which completely or partially reduced levels of PAI-1 are found include bleeding conditions (Schleef, R. R., et al., J. Clin. Invest. 83: 1747-1752 (1989); Fay, W. P., et al, N. Eng. J. Med. 327: 1729-1733 (1992); Liu, Y.-X., et al., Eur. J. Biochem. 195: 54-555, 1991).

A large body of experimental evidence from studies involving both model systems and human patients suggests that u-PA may play an important role in tumor biology and provides a compelling rationale to pursue the development of u-PA inhibitors. For example, anti-u-PA antibodies inhibit metastasis of HEp3 human carcinoma cells to chick embryo lymph nodes, heart, and lung (Ossowski, L., and Reich, E. (1983) Cell 35: 611-619), and similar studies demonstrated that these antibodies inhibit lung metastasis in mice following injection of B16 melanoma cells into the tail vein (Hearing, V. J., et al., (1988) Cancer Res. 48: 1270-1278). Anti-u-PA antibodies also inhibit both local invasiveness and lung metastasis in nude mice bearing subcutaneous MDA-MB-231 breast carcinoma tumors. In addition, a recent study indicated that u-PA deficient mice are resistant to the induction and/or progression of several tumor types in a two stage, chemical carcinogenesis model. Finally, high levels of tumor-associated u-PA correlate strongly with both a shortened disease-free interval and poor survival in several different human cancers (Duffy, M. J., et al., (1988) Cancer 62: 531-533; Janicke, F., et al., (1990) Fibrinolysis 4: 69-78; Duffy, M. J. (1993) Fibrinolysis 7: 295-302).

Because mice lacking either u-PA or t-PA do not develop thrombotic disorders, selective inhibition of either of these two enzymes seems unlikely to create thrombotic complications in vivo. On the other hand, mice lacking both u-PA and t-PA suffer severe thrombosis in many organs and tissues, resulting in a significantly reduced life expectancy. Nonselective inhibition of these two enzymes, therefore, seems almost certain to produce catastrophic consequences in the clinical setting. Consequently, significant interest exists in the development of inhibitors that are stringently specific for either t-PA or u-PA, which are expected to facilitate a detailed investigation of the precise roles of the two enzymes in several important pathological processes and may aid the development of novel therapeutic agents to combat these processes. Rational design of these selective inhibitors is greatly complicated, however, by the absence of obvious “lead compounds”; both their primary physiological substrate and inhibitors fail to discriminate between the two closely related proteases.

Combinatorial libraries provide a convenient means for screening a very large number of compounds. In general, combinatorial libraries provide a large number (10⁴-10⁸) of variants of small molecules such as peptides (Lam, K. S., Synthetic peptide libraries, in Meyers, R. A., Molecular Biology and Biotechnology. A Comprehensive Desk Reference, VCH Publishers, New York, 1995, pages 880-883). Combinatorial peptide libraries are large collections of different peptides, with many different possible combinations of amino acids joined together. The length of the peptides in the library can be chosen to suit the particular application.

The different molecules in a combinatorial library can be provided with a tag, such as an epitope recognized by an antibody, that may be used for identification and manipulation. Libraries in general can be constructed by synthesis of the different molecules on a substrate, or by a biological method, generally involving bacteriophages and bacteria.

Substrate bacteriophage display libraries have been used to elucidate optimal sub-site occupancy for substrates of t-PA and to isolate peptide substrates that were cleaved as much as 5300 times more efficiently by t-PA than peptides which contained the primary sequence of the actual target site present in plasminogen (Ding, C., et al., 1995). These selected substrates, however, were also efficiently cleaved by u-PA and therefore showed less than an order of magnitude preference for cleavage by t-PA compared to u-PA.

What is needed is a method for identifying substrates that show between about 10 fold to about 1,000 fold selectivity for one enzyme over another.

### SUMMARY OF THE INVENTION

The rational design of small molecule inhibitors as therapeutic agents is often complicated by the need to discriminate between binding to closely related enzymes. Appropriate selections of substrate phage can achieve this discrimination. Substrate subtraction libraries of the present invention provide substrates that can distinguish between any two distinct proteases. Multiple proteases can be used in the subtraction step to achieve even greater specificity. Moreover, both substrate and substrate subtraction libraries can be prepared as described herein for any enzymes that can use peptides or proteins as substrates.

The present invention creates substrate subtraction libraries that are useful in the identification of highly selective substrates for specific proteins and enzymes. The present invention is particularly useful in the identification of highly selective substrates for closely-related enzymes. Indeed, it is possible to prepare substrate subtraction libraries for any enzymes that use peptides or proteins as substrates. These techniques can be easily adapted to protein kineses, for example, by using antibodies against phosphoserine, phosphothreonine, or phosphotyrosine during the selection of substrate phage. Consequently, the construction and characterization of substrate and substrate subtraction libraries make substantial contributions to the rational design of highly specific, small molecule inhibitors of selected enzymes, a problem of paramount importance during the development of new therapeutic agents, and to provide key insights into the molecular determinants of specificity for a variety of important enzymes.

In one embodiment, the present invention provides a method for identifying the amino acid sequence of a peptide that is preferentially a substrate for a second enzyme. A combinatorial library having components that present corresponding peptides having random amino acid sequences of a chosen length is provided. The components of the combinatorial library are contacted with the first enzyme and the library separated into two portions, one that has peptides that were modified by the first enzyme and one that has peptides that were not. One or both portions of combinatorial library are contacted with the second enzyme, and the components of the combinatorial library that present corresponding peptides that were modified by contact with the one enzyme but not substantially modified by the other enzyme are identified. The sequences of the corresponding peptides that were modified by the one enzyme can then be determined. The process described is called “substrate subtraction screening” and the combinatorial libraries produced are called “substrate subtraction libraries.” The first enzyme and second enzyme are generally in the same broad class of enzymes, e.g., proteases, kinases, phosphatases, and the like. A suitable combinatorial library is a bacteriophage display library.

Another embodiment is a compound comprising the animo acid sequence determined by the method of substrate subtraction screening described above. Such compounds are useful as enzyme inhibitors.

Stringently specific small molecule inhibitors can not only be used to assess the individual roles of t-PA and u-PA during a wide variety of biological and pathological processes but also can provide important therapeutic benefits. Selective inhibition of u-PA can antagonize invasion, metastasis, and angiogenesis of specific tumors (Danø K., et al., Plasminogen activators, tissue degradation, and cancer. Adv. Cancer Res. 1985;44:139-266; Min, H. Y., et al., Urokinase receptor antagonists inhibit angiogenesis and primary tumor growth in syngeneic mice. Cancer Res. (1996);in press; Ossowski, L., Plasminogen activator dependent pathways in the dissemination of human tumor cells in the chick embryo. Cell 1988;52:321-328) as well as vascular re-stenosis following invasive procedures such as angioplasty (Carmeliet P, et al. Physiological consequences of loss of plasminogen activator gene function in mice. Nature 1994;368:419-424). Selective inhibition of t-PA can prevent specific types of neural degeneration (Strickland DK. Excitotoxin-induced neuronal degeneration and seizure are mediated by tissue plasminogen activator. Nature 1995 ;377 :340-344).

In preferred embodiments, the sequence determined by the method of substrate subtraction screening is incorporated in the construction of recombinant protease inhibitors, such as variants of PMI-1. In therapeutic embodiments, recombinant protease inhibitors, such as variants of PAI-1 are administered to a patient in an amount from about 0.003 to about 20 micrograms per kilogram body weight per day.

In another embodiment, antibodies to peptides identified using substrate subtraction libraries are also useful as an assay kit and method for detecting the level of active protease inhibitors. The antibodies may be used to assay the level of protease inhibitors, such as PAI-1, in a patient. The level of protease inhibitors, such as PAI-1, in a patient is a disease marker that is useful for predicting the development of a condition, identifying patients with the condition, predicting outcome of the condition, aiding timing and targeting of therapeutic interventions, and determining the pathogenesis of the condition in patients. Conditions in which the level of protease inhibitors, such as PAI-1, is a useful marker are bleeding conditions characterized by an inability to produce PAI-1 or a lack of active PAI-1. Antibodies to the reactive site of a serpin protease inhibitor such as PAI-1 would be useful for distinguishing between active and latent forms of the protease inhibitor.

In another embodiment, antibodies to peptides identified using substrate subtraction libraries are also useful to identify novel active protease inhibitors. The antibodies may be used to identify molecules having exposed sequences similar to the sequences of peptides identified using substrate subtraction libraries. This embodiment also is useful for screening naturally occurring compounds for protease inhibitor activity in the process of drug discovery.

In a further embodiment, antibodies to the identified peptides can be used for affinity purification of the peptides identified by the present invention. The peptides to be purified can be in a mixture of peptides or can be peptides produced by recombinant techniques.

Suitable antibodies comprise immunoglobulin molecules and immunologically active portions of immunoglobulin molecules, i.e., molecules that contain an antibody combining site or paratope. Exemplary antibody molecules are intact immunoglobulin molecules, substantially intact immunoglobulin molecules and those portions of an immunoglobulin molecule that contain the paratope, including those portions known in the art as Fab, Fab′ and F(ab′)₂.

### BRIEF DESCRIPTION OF THE DRAWINGS

In the Drawings,

FIG. 1 is a diagram depicting an outline of one protocol used to create substrate subtraction libraries, where the gene III fusion protein, phage, monoclonal antibodies, and immobilized protein A are not drawn to scale;

FIG. 2 is a diagram depicting an outline of another protocol used to create substrate subtraction libraries;

FIG. 3 is a representation of the results of a functional analysis of individual control or substrate phage stocks using a dot blot assay; and

FIG. 4 is a representation of the results of a functional analysis of specific cleavage of a fusion protein by t-PA.

### DETAILED DESCRIPTION OF PREFERRED EMBODIMENTS

The disclosed methods are useful general in the design of specific inhibitors of various proteins and enzymes. The method can be applied to other proteases, and to other classes of enzymes, including kinases and phosphatases. Thus, the disclosures relating to u-PA and t-PA should be considered exemplary and not limiting.

Examples of other suitable enzyme systems include proteases, including other serine proteases, as well as kinases and phosphatases.

The present invention relates to compositions including peptides and methods of identifying those peptides that are selectively reactive between a first enzyme and a second enzyme. A combinatorial library displaying different peptides is provided. This combinatorial library can be made, for example, by random mutation of bacteriophages displaying peptide sequences. The phage express the peptide sequences externally and, after reaction with the enzymes, the desired phage can be enriched. Alternatively, the combinatorial library can include an array of peptide substrates whose amino acid sequences are known by their location on the substrate. The production of such library arrays on substrates is well known in the art. See, for example, Meyers, Molecular Biology and Biotechnology: A Comprehensive Desk Reference, VCM Publishers, New York 1995, pages 880-883. The use of a phage library is preferred because it is generally able to a greater range of different sequences, usually on the order of 10⁸ different amino acid sequences.

The combinatorial library is contacted with the first enzyme to permit the first enzyme to modify some of the components of the combinatorial library. Those components which are modified by the first enzyme are then separated from the components of the library that are substantially unmodified by the first enzyme.

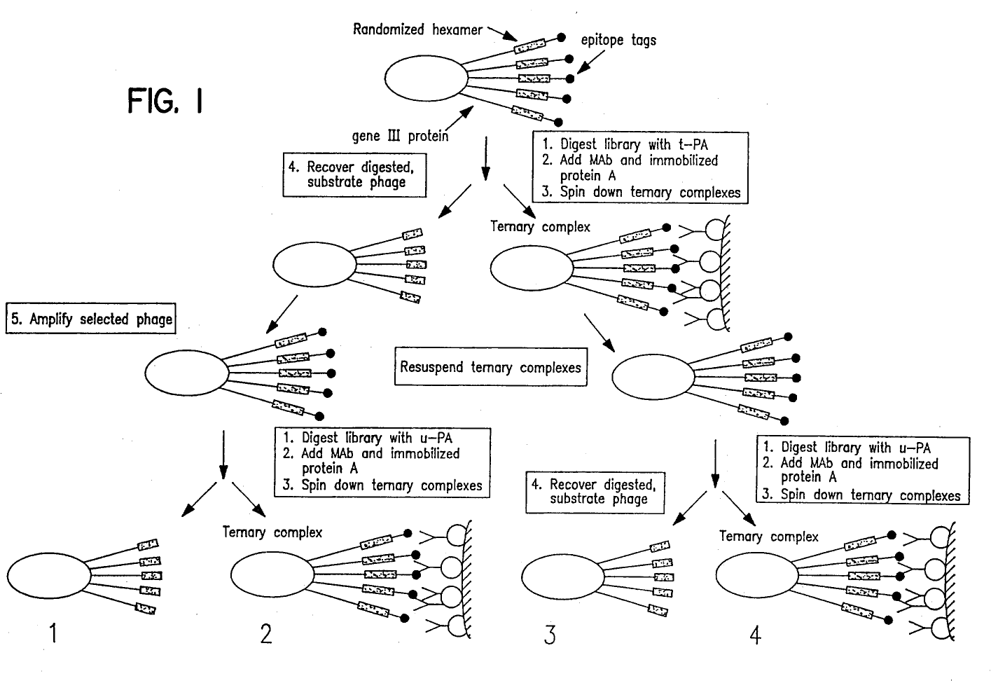

At that point, either one of two steps is then taken. The modified portion is contacted with the second enzyme or the unmodified portion is contacted with the second enzyme. It is then possible to identify at least some of the components of the combinatorial library that are modified by one enzyme but substantially unmodified by the other. A working example showing these pathways is demonstrated in FIG. 1. The example of phage displaying hexamer peptides with epitope tags on the end of the peptide is shown. The phage library is then allowed to contact the t-PA enzyme to digest the peptides. This results in the separation into two portions, those components which were modified by the t-PA are shown on the left and those not modified by the t-PA are shown on the right. As shown, separation is accomplished by immobilizing with monoclonal antibodies.

The phage that were modified by t-PA is then amplified to produce phage displaying the entire peptide and epitope tabs. As shown on the right, the phage were then digested with u-PA and the phage which were modified were separated from unmodified phage by the use of monoclonal antibodies. This results in a first population of phage expressing peptides that react with both t-PA and u-PA. It also results in a second population that reacts with t-PA but not u-PA. It is the second population that is of interest in the present invention. This population can be resuspended and amplified.

Alternatively shown on the left side of FIG. 1, the population of phage that was unmodified by t-PA are resuspended and contacted with u-PA. After digestion, monoclonal antibodies are used as before. This results in two more populations, a third population which includes phage expressing peptides that react with u-PA but not t-PA, and a fourth population which includes phage that did not react with either u-PA or t-PA. It is the third population which is also of interest in the present invention. This population can then be amplified.

As can be seen, both of these routes allow the identification of the some of the components of the combinatorial library that are modified by one enzyme but not modified by the other. As shown in FIG. 1, these are the third and fourth populations. The entire procedure or individual screening steps can be repeated one or more times to increase the selectivity.

At least some of the components that are modified by one enzyme but are not modified by the other enzyme are then identified. In the case of the phage library, phage in the second and third populations are identified in this manner. The phage can then be grown in culture allowing the identification to also include determining the amino acid sequence of at least one of the displayed peptides. The resulting peptides have a selectivity preferably of at least 10 fold, and more preferably of at least 50 fold, for the desired enzyme compound to the undesired enzyme. The enzymes are preferably both proteases such as kinases and phosphatases.

In the case of library arrays on substrates, portions of the library, such as the use of two identical arrays, are individually reacted with each of the enzymes. The location of peptide cleaving is determined, such as by use of a cysteine residue or an epitope tag and labeled antibodies on each array and the results compared to determine which peptides react with one enzyme but not the other.

The amino acid sequence or sequences that are determined can then be used to make peptides having these sequences. These peptides can be used to prepare antibodies as is known in the art and used to purify recombinant peptides or identify naturally occurring protease inhibitors which are immunoreactive with the antibodies so produced. The antibodies are also used for diagnostic assays which can distinguish between active and latent forms of the protease inhibitors.

The amino acid sequences determined can be used to engineer protease inhibitors. For example, the amino acid sequence determined to be highly selective toward to the second enzyme can be used in the design of an inhibitor for the second enzyme which has a low reactivity with the first enzyme. This allows the treatment of medical conditions where targeting of inhibition of the second enzyme is useful, while inhibiting the first enzyme is not desired.

Such inhibitors would have a structure corresponding to naturally occurring enzyme inhibitors. The amino acid sequence of such inhibitors would be modified to include the amino acid sequence taught by the method of this invention. Alternatively, substrates including amino acid sequences as taught by the present invention are modified to create inhibitors.

The amino acid sequence, together with the other coding for the inhibitor is then coded on a plasmid or other DNA vector for introduction into a prokaryotic or eukaryotic cell as is well known in the art. Such cells will produce the desired enzyme inhibitor which can be purified using the antibodies discussed above.

A substrate subtraction combinatorial library can also be produced. The combinatorial library is contacted with the first enzyme to modify some of the components of the combinatorial library. The phage can then be separated by solid phase or precipitation as known in the art with either population serving as a substrate substraction library. The example of the first enzyme will be used.

Such a substrate subtraction library substantially lacks peptides that are effective substrates for the first enzyme, meaning that the peptides have a reactivity of less than about 10 percent of the best naturally occurring molecule that the first enzyme reacts with. For example, in the case of u-PA as shown in Table 5 below, the native or wild type PAI-1 has a rate constant of 1.9×10⁷M⁻¹s⁻¹ while the selected PPAI-1/P3R has a rate constant of 1.0×10⁵ meaning that the reactivity is less than 1 percent. Table 8 also makes similar comparisons with the wild type. The peptides in the combinatorial library preferably have a k_{cat}/Kₘ ratio of less than about 500 M⁻¹s⁻¹ and preferably a k_{cat}/Kₘ ratio of less than about 100 M⁻¹s⁻¹.

While the removal of the peptides that are effective substrates for the first enzyme is preferred for preparing a substrate subtraction library, it is not necessary for practicing the invention. Depending on the enzyme involved, the method for the present invention can be practiced with peptides having a reactivity even greater than 10% when compared to the naturally occurring molecule.

The present invention also provides for therapeutic treatment of a patient. This treatment can take two general forms. The determined peptide itself can be administered to the patient to in effect overload the patient's enzymes thereby creating an inhibitory effect. In such cases, the preferred administration rates of the peptides, such as t-PA and u-PA, are from about 0.1 micrograms/kg to about 50 micrograms/kg.

Alternatively, enzyme inhibitors made according to the present invention can also be administered to the patient. These inhibitors directly inhibit the activity of the enzymes. In the case of u-PA and t-PA, the preferred administration rates are from about 0.003 micrograms/kg to about 20 micrograms/kg.

Some of the diseases that can be effectively treated are serpin deficiencies such as pulmonary emphysema, associated with deficiencies of α₁-proteinase inhibitor, antithrombin deficiency, hereditary angioedema associated with deficiencies of Cl- inhibitor, bleeding disorders associated with deficiencies in α-antiplasmin or PAI-1 (Gettins et al. 1996). Serpins have also been implicated in several forms of cancer, including squamous cell carcinoma (Gettins, et al. 1996). In each case, a physiologically effective amount of the peptide or the enzyme inhibitor is administered. Examples of specific peptides are discussed below.

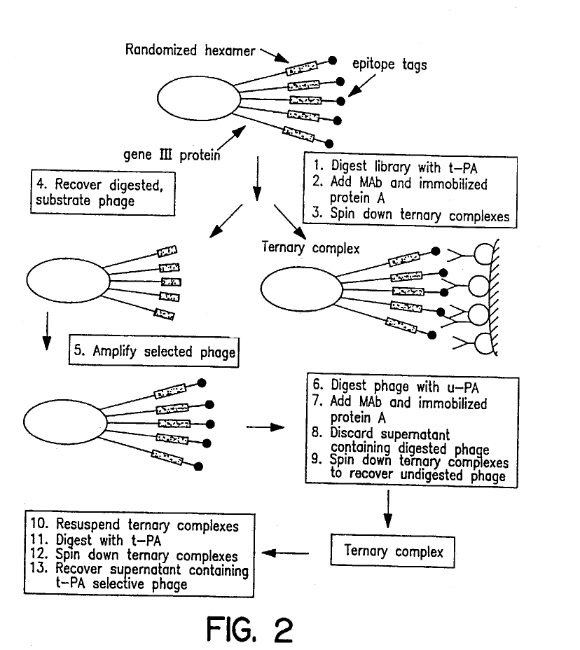

Referring to FIG. 2, the specific example of phage having hexamer peptides and epitope tags are disclosed. This example pre-processes the library to enhance selectivity. The phage library is then contacted with the t-PA enzyme and digested. Monoclonal antibodies to the epitope tags are then added to separate cleaved and uncleaved expressed peptides. The phage having cleaved peptides are then selected and amplified. The amplified phage are then again digested with u-PA. Monoclonal antibody to the epitope tags is again used to immobilize the phage having peptides that were not modified by the u-PA. The undigested phage are then recovered and resuspended. They are again digested with t-PA and the unreacted phage are again separated using monoclonal antibodies. The phage which are again reacted with t-PA are then identified in the supernatant.

### EXAMPLE 1

### Preparation and Use of Substrate Subtraction Libraries Reagents

Competent MC1061 (F−) E. coli and nitrocellulose were purchased from Bio-Rad Laboratories. Pansorbin (Protein A-bearing S. aureus) cells were obtained from Calbiochem (San Diego, Calif.). K91 (F+) and MCI061 (F−) strains of E. coli were provided by Steve Cwirla (Affymax). MAb 3-E7 was purchased from Gramsch Laboratories (Schwabhausen, FRO). u-PA was obtained from Jack Henkin (Abbott Laboratories).

A polyvalent fd phage library that displayed random hexapeptide sequences and contained 2×10⁸ independent recombinants was prepared (Ding, C., et al., 1995; Smith, M. M., et al, 1995). Peptides were synthesized and purified as described (Madison, E. L., et al. (1995) J. Biol. Chem. 270,7558-7562.). Each member of this library displayed an N-terminal extension from phage coat protein III (pIII) containing a randomized region of six amino acids fused to pIII, followed by a six residue linker sequence (SSGGSG) and the epitopes for mAb 179 and mAb 3-E7. Because neither t-PA or u-PA digests the pIII sequence, the antibody epitopes, or the flexible linker sequence, the loss of antibody epitopes from the phage surface upon incubation with either enzyme requires cleavage of the randomized peptide insert. Incubation of the library with t-PA, followed by removal of phage retaining the antibody epitopes, therefore, accomplishes the enrichment of phage clones whose random hexamer sequence can be cleaved by t-PA.

The detailed construction of the phage vector fAFFI-tether C (fTC) and the random hexapeptide library fAFF-TC-LIB has been previously described (Smith, M. M., et al, 1995). Control substrate phage frC-PL, which contained the physiological target sequence for u-PA and t-PA, was constructed by hybridizing the single stranded oligonucleotides 5′-TCGAGCGGTGGATCCGGTACTGGTCGTACTGGTCATGCTCTGGTAC-3′ and 5′-CGCCACCTAGGCCAGGACCAGCACAACAACCACGAGAC-3′ and then ligating the annealed, double stranded products into the Xho I/Kpn I-cut vector frC. All constructs were first transformed into MC1061 by electroporation and then transferred into K91.

Measurement of Enzyme Concentrations.

Concentrations of functional t-PA and u-PA were measured by active site titration with 4-methylumbelliferyl p-guanidinobenzoate (Jameson, G., et al., (1973) Biochem. J. 131, 107-117) using a Perkin-Elmer LS 50B Luminescence Fluorometer as previously described (Madison, E. L., et al., (1995) J. Biol. Chem. 270: 7558-7562). In addition, the enzymes were titrated with a standard PAI-1 preparation that had been previously titrated against a trypsin primary standard. Total enzyme concentrations were measured by ELISA.

The procedure used in producing substrate substraction libraries is outlined in FIG. 2. The initial phage library was subjected to three rounds of high stringency selection with t-PA to assure the preparation of an intermediate library that is highly enriched for phage that are very good substrates of t-PA. This intermediate library was then digested at low stringency with u-PA to remove phage that are moderate or good substrates for u-PA. Substrate subtraction was accomplished after the protease digestion of phage by adding Mab 3E-7 and immobilized protein A (Pansorbin cells, Calbiochem, San Diego, Calif.) to the reaction mixture and precipitating the ternary complexes which contain the undigested phage.

In contrast to earlier three rounds of selections, phage remaining in solution were then discarded, and the precipitate containing the ternary complexes is resuspended. Phage that were preferentially cleaved by t-PA were then identified by their release from the ternary complexes by digestion at high stringency with t-PA.

Preparation and Seguencing of DNA from Phaze Clones.

DNA samples were prepared from identified phage clones as previously described (Ding, C., et al., (1995)). Briefly, phage are precipitated from a 1 ml overnight culture by adding 200 μl of 20% polyethylene glycol in 2.5 M NaCl. The mixture was incubated on ice for 30 min., and the phage pellet was collected by microcentrifugation for 5 min. The phage were resuspended in 40 μl lysis buffer (10 mM Tris-HCL, pH 7.6, 0.1 mM EDTA, 0.5% Triton X-100) and heated at 80° C. for 15 min. Single stranded DNA was purified by phenol extraction and ethanol precipitation and sequenced according to the method of Sanger.

The kinetic analysis of particular clones is summarized in Table 1. Tables 2 and 3 summarize the sequences of other additional clones. Table 2 shows the sequences of 37 t-PA—selective phage clones isolated and functionally verified containing 32 distinct substrate sequences. For comparison, the sequences of six u-PA selective clones are listed in Table 2.

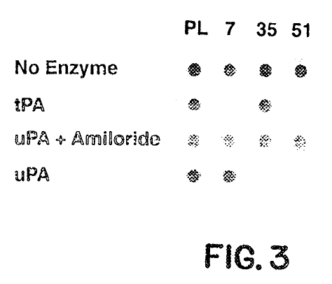

To verify that the substrate subtraction library had yielded substrates that were preferentially cleaved by t-PA, digestion of individual phage stocks by t-PA and u-PA was analyzed by a dot blot assay that was performed as previously described (Ding, L., et al., 1995; Smith, M. M., et al., 1995) (FIG. 3). Loss of positive staining indicates loss of antibody epitopes from the phage due to proteolytic cleavage of the random hexamer region. Control phage PL contains the P3- P3′ region of the actual target sequence present in plasminogen (PGRVVG, residues 4-9 of SEQ ID NO:1) and was not digested by either enzyme under the conditions used in this test. Substrate phage 51 contained the hexamer RIARRA (SEQ ID NO:148) and was a substrate of both t-PA and u-PA. Phage 7 contained the hexamer FRGRAA (SEQ ID NO:25) and was a t-PA selective substrate. Phage 33 contained the hexamer RSANAI (SEQ ID NO:51) and was a u-PA selective substrate.

Kinetics of Cleavage of Synthetic Peptides by t-PA and u-PA.

Individual phage stocks were prepared and digested with no enzyme, t-PA, u-PA, or u-PA in the presence of 1 mM amiloride, a specific inhibitor of u-PA. Kinetic-data were obtained by incubating various concentrations of peptide with a constant enzyme concentration to achieve between 5 and 20% cleavage of the peptide in each reaction. For assays with u-PA, enzyme concentration was either 815 or 635 nM. For assays with t-PA enzyme concentration was 700 nM. Peptide concentrations were chosen where possible to surround Kₘ and in all cases were between 0.5 and 32 mM. The buffer used in these assays has been described (Madison, E. L., et al., 1995). Reactions were stopped by addition of triflouroacetic acid to 0.33% or by freezing on dry ice. Cleavage of the 13 and 14 residue peptides was monitored by reverse phase HPLC as described (Madison, E. L., et al., 1995). The 4-6 residue peptides were acylated at their amino termini and amidated at their carboxyl termini. Cleavage of the 4-6 residue peptides was monitored by hydrophilic interaction HPLC chromatography (HILIC) (Alpert, A. J. (1990) J. Chromatog. 499, 177-196.) using a polyhydroxyaspartamine column from PolyLC (Columbia, Md.). Buffer A was 50 mM triethylamine phosphate in 10% acetonitrile and buffer B was 10 mM triethylamine phosphate in 80% acetonitrile. Peptides were eluted by a gradient which was varied from 100% Buffer B to 100% Buffer A during a 13 minute interval. The percent of cleaved peptide was calculated by dividing the area under the product peaks by the total area under substrate and product peaks. For all peptides containing multiple basic residues, mass spectral analysis of products confirmed that cleavage occurred at a single site and identified the scissile bond. Data were interpreted by Eadie-Hofstee analysis. Errors were determined as described (Taylor, J. R., 1982) An introduction to error analysis. The study of uncertainties in physical measurements. University Science Books, Mill Valley, Calif.) and were <25%.

The results of kinetic analysis are summarized in Table 1, below, which compares the values determined for cleavage of the native target, plasminogen (I), t-PA selective peptides (II-X) and u-PA selective peptides (XI-XVIII).

Three peptide substrates (II-IV) containing hexamer sequences present in individual members of the substrate subtraction library were synthesized and characterized to provide a quantitative analysis of the properties of putative t-PA selective substrates. These peptides were cleaved between 13 and 47-fold more efficiently by t-PA than by u-PA (Table 1).

Comparison of the hexamer sequences obtained from the substrate subtraction library and the consensus sequences derived for substrates of u-PA and t-PA confirms the expected intimate similarity between optimal sub-site occupancy for these two closely related enzymes. In addition, these data strongly suggest that the P3 residue of a substrate is the primary determinant of the ability to distinguish between t-PA and u-PA. t-PA prefers arginine or large hydrophobic residues at this position while u-PA favors small hydrophilic residues, particularly serine.

In contrast to results obtained using t-PA, standard phage display was sufficient to yield highly selective u-PA substrates. One hundred substrate phage, containing 89 distinct random hexamer sequences, were selected using u-PA (Table 4). Dot blot analysis of the individual phage stocks under increasingly stringent conditions indicated that eleven clones, containing eight distinct hexamer sequences, were particularly labile u-PA substrates (Table 3). Peptides containing four of these eight hexamer sequences (XI-XIV) were synthesized and characterized. All four peptides were substantially improved substrates for u-PA, by factors of 840-5300, compared to a control peptide (I) that contained the actual target sequence present in plasminogen (Table 3). The four peptides were also cleaved 16-89 times more efficiently by u-PA than by t-PA.

To confirm the key role of P3 in defining specificity differences between t-PA and u-PA, variants of a u-PA selective peptide that contained either tyrosine or arginine at this position were synthesized and characterized. In striking contrast to parent peptide XI, the two variants (peptides VIII and IX) were cleaved 5.2-5.7 times more efficiently by t-PA than by u-PA, a 320-fold reversal in substrate preference (Table 1). Further replacement of the glycine found at P4 of the u-PA selective substrate with glutamine (peptide X) increased t-PA selectivity to 19-fold over u-PA. Point mutations at both P4 and P3, therefore, altered the relative specificity of t-PA versus u-PA by a factor of 1200.

The kinetic analysis described above was performed using substrate peptides that were 14 amino acids in length. To confirm that the specificity we observed was inherent in the selected hexapeptide sequences, and therefore would be expected to be readily converted into viable, small molecule peptidometics, we examined the kinetics of cleavage of short peptides containing only sequences found within selected hexapeptide sequences. For both t-PA and u-PA selective substrates, specificity was maintained by related pentapeptides. The peptide FRGRK was cleaved approximately 74 times more efficiently by t-PA than by u-PA while the peptide GSGKS was hydrolyzed approximately 120 times more efficiently by u-PA than by t-PA (Table 1). The relative specificity of these two pentapeptides for cleavage by t-PA versus u-PA, therefore, differs by a factor of approximately 9000, indicating that appropriate occupancy of the P4-P1′ sub-sites alone can mediate the ability of a substrate to distinguish the closely related enzymes t-PA and u-PA.

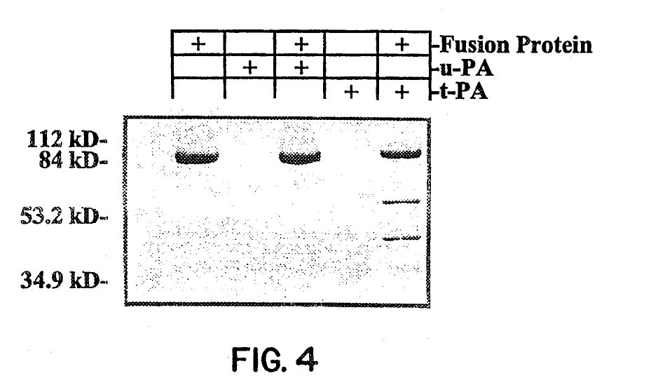

To define further the extent of substrate discrimination that could be achieved in other structural contexts, the t-PA selective hexapeptide QRGRSA was introduced into a fusion protein consisting of a photoreceptor protein linked to maltose binding protein. t-PA readily cleaved the fusion protein (FIG. 4) whereas u-PA did not, demonstrating the maintenance of specificity for cleavage of this selected, primary sequence in the structural context of a protein substrate.

The fusion consists of maltose binding protein fused to the amino terminus of the HY4 gene product of Arabidopsis thaliana with a linking region in between coding for the amino acid sequence QRGRSA, which is cleaved by t-PA between R and S. The concentration of fusion protein in each lane is 1.4 mM and the concentration of u-PA or u-PA is 150 nM. The reaction buffer contains 50 mM Tris pH 7.5, 0.1 M NaCl, 1 mM EDTA, and 0.01% Tween 20. Reactions were set up in a total volume of 20 mL and allowed to incubate 16 hours at 25 C. and then stopped by the addition of 5 mL of 5× loading buffer and separated by electrophoresis on 12% polyacrylamide. The gel was stained with Coomassie Brilliant Blue 30 minutes and de-stained overnight.

TABLE 1

Comparison of k_{cat}, and Kₘ and k_{cat}/Kₘ for hydrolysis of peptides
selected for preferential cleavage by t-PA or u-PA

| | |
|---|---|
| t-PA | u-PA |

| | | | | | | | | |
|---|---|---|---|---|---|---|---|---|
| Substrate | SEQ ID | k_{cat} | Kₘ | k_{cat}/Kₘ | k_{cat} | Kₘ | k_{cat}/Kₘ | t-PA:u-PA |
| ¹(Pn,.P3,P2,P1,↓P1′,P2′,P3′..Pn) | NO: | s⁻¹ | (μM) | (M⁻¹s⁻¹) | s⁻¹ | (μM) | (M⁻¹s⁻¹) | Selectivity |

Native cleavage sequence from Plasminogen

| | | | | | | | | | |
|---|---|---|---|---|---|---|---|---|---|
| (I) | KKSPGR↓WGGSVAH | 1 | 0.0043 | 15000 | 029 | 0.003 | 3400 | 0.88 | 0.33 |

t-PA selective peptides

| | | | | | | | | | |
|---|---|---|---|---|---|---|---|---|---|
| (II) | LGGSGQRGR↓KALE | 2 | 0.99 | 2300 | 430 | 0.02 | 2180 | 9.2 | 47 |
| (III) | LGGSGERAR↓GALE | 3 | 0.073 | 1410 | 52 | 0.004 | 970 | 4.0 | 13 |
| (IV) | LGGSGHYGR↓SGLE | 4 | 1.29 | 4010 | 322 | 0.059 | 3800 | 15 | 21 |
| (V) | YGR↓S | 5 | 23.7 | 6000 | 3950 | 2.6 | 11400 | 230 | 17 |
| (VI) | RGR↓K | 6 | 15.3 | 16600 | 992 | 0.76 | 46500 | 16 | 57 |
| (VII) | FRGR↓K | 7 | 12.2 | 9800 | 1240 | 0.14 | 8600 | 16 | 78 |
| (VIII) | LGGYGR↓SANAILE | 8 | 3.29 | 1850 | 1800 | 0.7 | 2200 | 318 | 5.7 |
| (IX) | LGGRGR↓SANAILE | 9 | 0.85 | 2400 | 350 | 0.08 | 1200 | 67 | 5.2 |
| (X) | LGQRGR↓SANAILE | 10 | 2.55 | 3000 | 850 | 0.068 | 1500 | 45 | 19 |

u-PA selective peptides

| | | | | | | | | | |
|---|---|---|---|---|---|---|---|---|---|
| (XI) | LGGSGR↓SANAlLE | 11 | 0.305 | 4080 | 75 | 2.83 | 603 | 4700 | 0.016 |
| (XII) LGGSGR↓NAQVRLE | 12 | 0.255 | 7000 | 36 | 3.69 | 1160 | 3200 | 0.011 | |
| (XIII) | LGGSGR↓SATRDLE | 13 | 0.068 | 1500 | 45 | 0.54 | 733 | 740 | 0.06 |
| (XIV) | LGGSGR↓KASLSLE | 14 | 0.168 | 5100 | 33 | 1.14 | 1130 | 1010 | 0.032 |
| (XV) | SGR↓S | 15 | 5.0 | 15000 | 330 | 2.3 | 2100 | 1100 | 0.30 |
| (XVI) | SGR↓SA | 16 | 2.4 | 40000 | 60 | 3.7 | 3100 | 1200 | 0.05 |
| (XVII) | SGK↓S | 17 | 0.19 | 28000 | 6.8 | 1.22 | 7900 | 154 | 0.04 |
| (XVIII) | GSGK↓S | 18 | 0.07 | 44000 | 1.6 | 0.82 | 4250 | 193 | 0.008 |

¹Posltional nomenclature of subsite residues. Arrows denote the position of peptide bond hydrolysis. The peptide bond is cleaved between P1 and P1′. The error in these determinations was 4-22%.

TABLE 2

Primary sequences of hexamers of t-PA selective
substrate clones

| | | | | | | | | | | | |
|---|---|---|---|---|---|---|---|---|---|---|---|
| SEQ ID | Clone | P5 | P4 | P3 | P2 | P1 | P1′ | P2′ | P3′ | P4′ | P5′ |

| | | | | | | | | | |
|---|---|---|---|---|---|---|---|---|---|
| 19 | 1 | | | A | L | R | R | G | D |
| 20 | 2 | D | Y | R | G | R | M | (L) | |
| 21 | 3 | | E | R | A | R | G | A | |
| 22 | 4 | | E | R | L | R | K | A | |
| 23 | 5 | | | F | G | R | H | A | A |
| 24 | 6 | | F | L | P | R | T | A | |
| 25 | 7 | | F | R | G | R | A | A | |
| 26 | 8 | | H | R | M | R | M | G | |
| 27 | 9 | | H | Y | G | R | S | G | |
| 28 | 10 | | | I | M | R | R | G | K |
| 29 | 11 | I | T | Y | G | R | R | (L) | |
| 30 | 12 | | K | F | T | R | S | G | |
| 31 | 13 | | L | I | P | R | R | A | |
| 32 | 14 | M | T | R | K | R | M | (L) | |
| 33 | 15 | | N | F | A | R | M | G | |
| 34 | 16 | | N | H | L | R | K | A | |
| 35 | 17 | | N | V | G | R | M | G | |
| 36 | 18 | | N | V | S | R | R | G | |
| 37 | 19 | | P | I | S | R | R | A | |
| 38 | 20 | | P | V | G | R | M | G | |
| 39 | 21 | | Q | R | G | R | K | A | |
| 40 | 22 | | R | L | L | R | S | V | |
| 41 | 23 | | S | F | G | R | R | H | |
| 42 | 24 | S | L | R | G | R | S | (L) | |
| 43 | 25 | | T | V | L | R | R | A | |
| 44 | 26 | | | V | A | R | R | V | K |
| 45 | 27 | | V | I | A | R | S | N | |
| 46 | 28 | | V | N | T | K | S | G | |
| 47 | 29 | | V | R | A | R | G | A | |
| 48 | 30 | V | R | R | G | R | S | (L) | |
| 49 | 31 | | V | R | R | R | G | A | |
| 50 | 32 | | T | R | V | R | A | K | |

TABLE 3

Primary sequences of hexamers of most labile u-PA
substrate clones

| | | | | | | | | | | | |
|---|---|---|---|---|---|---|---|---|---|---|---|
| SEQ ID | Clone | P5 | P4 | P3 | P2 | P1 | P1′ | P2′ | P3′ | P4′ | P5′ |

| | | | | | | | | | |
|---|---|---|---|---|---|---|---|---|---|
| 51 | 33 | (S | G) | R | S | A | N | A | I |
| 52 | 34 | (S | G) | R | N | A | Q | V | R |
| 53 | 35 | (S | G) | R | S | A | T | R | D |
| 54 | 36 | (S | G) | R | S | A | K | V | D |
| 55 | 37 | (S | G) | R | K | A | S | L | S |
| 56 | 38 | (S | G) | R | R | A | V | S | N |
| 57 | 39 | (S | G) | R | S | A | V | V | K |
| 58 | 40 | (S | G) | R | S | S | S | S | H |

TABLE 4

u-PA-Phage Selection Summary

| | | | | | | | | | | | |
|---|---|---|---|---|---|---|---|---|---|---|---|
| SEQ | | | | | | | | | | | |
| ID | SELECTIVITY | P5 | P4 | P3 | P2 | P1 | P1′ | P2′ | P3′ | P4′ | P5′ |

| | | | | | | | | | | | |
|---|---|---|---|---|---|---|---|---|---|---|---|
| 59 | (U,T) | | A | I | K | R | S | A | | | |
| 60 | (U) | | | | G | R | R | G | N | R | |
| 61 | (U) | | | | G | R | S | V | N | N | |
| 62 | (U) | | | H | T | R | R | M | K | | |
| 63 | (U,T) | I | S | T | A | R | M | | | | |
| 64 | (U) | | | | | K | A | A | D | V | T |
| 65 | (U) | | | | | K | K | R | T | N | D |
| 66 | (U) | K | M | S | A | R | I | | | | |
| 67 | (U) | | | | K | R | R | D | V | A | |
| 68 | (U) | | | | K | R | V | S | K | N | |
| 69 | (U) | | | | | K | S | A | D | A | A |
| 70 | (U) | | | | | R | A | A | A | M | V |
| 71 | (U) | | | | | R | A | G | N | I | R |
| 72 | (U) | | | | | R | A | H | R | D | N |
| 73 | (U) | | | | | R | A | R | D | D | R |
| 74 | (U) | | | | | R | A | R | H | M | V |
| 75 | (U) | | | | | R | A | R | S | P | R |
| 76 | (U) | | | | | R | A | V | G | H | Q |
| 77 | (U) | | | | | R | A | V | V | D | S |
| 78 | (U) | | | | | R | G | G | K | G | P |
| 79 | (U,T) | | | | | R | G | R | S | A | V |
| 80 | (U) | | | | | R | G | V | D | M | N |
| 81 | (U) | | | | | R | G | V | K | M | H |
| 82 | (U) | | | | | R | H | R | S | D | I |
| 83 | (U) | | | | | R | K | G | Q | G | G |
| 84 | (U) | | | | | R | K | L | H | M | N |
| 85 | (U) | | | | | R | K | M | D | M | G |
| 86 | (U) | | | | | R | K | M | D | R | S |
| 87 | (U) | | | | | R | K | M | R | M | G |
| 88 | (U) | | | | | R | K | N | Q | R | V |
| 89 | (U) | | | | | R | K | Q | R | D | S |
| 90 | (U) | | | | | R | K | R | V | G | A |
| 91 | (U) | | | | | R | K | S | K | V | V |
| 92 | (U) | | | | | R | K | S | T | S | S |
| 93 | (U) | | | | | R | K | V | G | S | L |
| 94 | (U) | | | | | R | K | V | P | G | S |
| 95 | (U) | | | | | R | K | W | I | S | G |
| 96 | (U) | | | | | R | L | A | T | K | A |
| 97 | (U) | | | | | R | M | R | K | N | D |
| 98 | (U) | | | | | R | N | A | Q | V | R |
| 99 | (U,T) | | | | | R | N | A | V | E | P |
| 100 | (U) | | | | | R | N | D | R | L | N |
| 101 | (U) | | | | | R | N | G | K | S | R |
| 102 | (U) | | | | | R | N | M | P | L | L |
| 103 | (U) | | | | | R | N | T | G | S | H |
| 104 | (U) | | | | | R | R | M | T | M | G |
| 105 | (U) | | | | | R | R | R | L | N | M |
| 106 | (U) | | | | | R | R | T | L | D | F |
| 107 | (U,T) | | | | | R | S | A | K | V | D |
| 108 | (U) | | | | | R | S | A | N | A | I |
| 109 | (U) | | | | | R | S | A | T | R | D |
| 110 | (U) | | | | | R | S | A | V | V | K |
| 111 | (U) | | | | | R | S | D | Q | F | L |
| 112 | (U) | | | | | R | S | D | N | P | N |
| 113 | (U) | | | | | R | S | E | R | S | L |
| 114 | (U) | | | | | R | S | G | D | P | G |
| 115 | (U) | | | | | R | S | G | N | T | T |
| 116 | (U) | | | | | R | S | G | N | M | G |
| 117 | (U) | | | | | R | S | N | G | V | G |
| 118 | (U) | | | | | R | S | P | D | G | M |
| 119 | (U) | | | | | R | S | R | R | L | P |
| 120 | (U) | | | | | R | S | R | V | T | S |
| 121 | (U) | | | | | R | S | S | H | S | S |
| 122 | (U) | | | | | R | S | S | Q | A | A |
| 123 | (U) | | | | | R | S | S | S | S | H |
| 124 | (U) | | | | | R | S | S | S | T | V |
| 125 | (U) | | | | | R | S | T | D | L | G |
| 126 | (U) | | | | | R | S | T | N | V | E |
| 127 | (U) | | | | | R | S | T | R | H | K |
| 128 | (U) | | | | | R | S | Y | T | N | S |
| 129 | (U) | | | | | R | T | S | P | S | T |
| 130 | (U) | | | | | R | T | S | V | N | L |
| 131 | (U) | | | S | G | R | A | R | Q | | |
| 132 | (U) | | | S | K | R | A | S | I | | |
| 133 | (U) | | | | S | K | S | G | R | S | |
| 134 | (U) S | Q | T | C | V | R | | | | | |
| 135 | (U) | | | S | S | R | N | A | D | | |
| 136 | (U) | | | T | A | R | L | R | G | | |
| 137 | (U) | | | T | A | R | S | D | N | | |
| 138 | (U) | | | T | E | R | R | V | R | | |
| 139 | (U) | | | T | Q | R | S | T | G | | |
| 140 | (U) | | | | T | R | R | D | R | I | |
| 141 | (U) | | | T | S | R | M | G | T | | |
| 142 | (U) | | | T | S | R | Q | A | Q | | |
| 143 | (U) | | | T | T | R | R | N | K | | |
| 144 | (U) | | T | T | S | R | R | S | | | |
| 145 | (U,T) | | | V | A | R | M | Y | K | | |
| 146 | (U,T) | | | V | S | R | R | N | M | | |
| 147 | (U,T) | | W | S | G | R | S | G | | | |

### EXAMPLE 2

### Preparation of Specific Inhibitors of t-PA

Specific inhibitors of t-PA were designed using the sequences derived in Example 1 by making variants of the PAI-1, natural inhibitor of t-PA.

Site-directed Mutagenesis and Construction of an Expression Vector Encoding a Recombinant Variant of PAI-1.

The expression vector pPAIST7HS was derived from the plasmid pBR322 and contained a full length cDNA encoding human PAI-1 that was transcribed from a T7 gene 10 promoter (Tucker, H. M., et al., (1995) Nature Struct. Biol. 2:442-445). The 300 bp Sal I/Bam HI fragment of human PAI-1 was subcloned from pPAIST7HS into bacteriophage M13mp18. Single stranded DNA produced by the recombinant M13mp18 constructs was used as a template for site specific mutagenesis according to the method of Zoller and Smith (Zoller, M. I., and Smith, M. (1984) DNA 3, 479-488) as modified by Kunkel (Kunkel, T. A. (1985) Proc. Natl. Acad. Sci. U.S.A. 82, 488-492).

Expression of wild type and the mutated variant of PAI-1 was accomplished in the E. coli strain BL21[DE3]pLys^{s} (Novagen, Madison, Wis.) which synthesizes T7 RNA polymerase in the presence of isopropylothio-B-D-galactoside (IPTG). Bacterial cultures were grown at 37 degrees Celsius with vigorous shaking to an A₅₉₅ of 0.9-1.1, and IPTG was added to a final concentration of 1 mM to induce the synthesis of T7 RNA polymerase and the production of PAl-1 proteins. Cultures were grown for an addition 1-2 hrs at 37 degrees Celsius and then shifted to 30 degrees Celsius for 2-6 hours.

Cells were pelleted by centrifugation at 8000×g for 20 min at 4 degrees Celsius and resuspended in 40 ml of cold start buffer (20 mM Sodium Acetate, 200 mM NaCl and 0.01% Tween 20, pH 5.6). The cell suspension was disrupted in a French pressure cell (Aminco), and cellular debris was removed by ultracentrifugation for 25 min at 32000×g.

Purification of soluble, active PAl-1 was performed as previously described (Sancho, E., et al., (1994) Eur. J. Biochem. 224:125-134). PAI-1 containing supernatants were injected onto a XK-26 column (Pharmacia Biotech) packed with CM-50 Sephadex (Pharmacia). The column was washed with 5 column volumes of start buffer (20 mM Sodium Acetate, 200 mM NaCl and 0.01% Tween 20, pH 5.6), and PAI-1 proteins were eluted using a 0.2 M-1.8 M linear gradient of NaCl in the same buffer. Peak fractions were collected, pooled, and concentrated using a Centriplus 30 concentrator (Amicon). Purified preparations were analyzed by activity measurements using standard, direct assays of t-PA, SDS-PAGE, and measurement of optical density at 280 nm.

Measurement of Active PAI-1 in Purified Preparations.

A primary standard of trypsin was prepared by active site titration using p-nitrophenyl-guanidinobenzoate HCl as described previously (Chase, T., and Shaw, E. (1967) Biochem. Biophys. Res. Commun. 29:508-514). Concentrations of active molecules in purified preparations of wild type or mutated PAI-1's were determined by titration of standardized trypsin as described by Olson et al. (Olson, S. T., et al., (1995) J. Biol. Chem. 270: 30007-30017) and by titration of standardized t-PA preparations.

Kinetic Analysis of the Inhibition of t-PA and u-PA by Recombinant PAI-1 and PAI-1/UKI.

Second order rate constants (kᵢ) for inhibition of t-PA or u-PA were determined using pseudo-first order (kᵢ<2×10⁶) or second order (kᵢ>2×10⁶) conditions. For each reaction, the concentrations of enzyme and inhibitor were chosen to yield several data points for which the residual enzymatic activity varied between 20%-80% of the initial activity. Reaction conditions and data analysis for pseudo-first order reactions were as previously described.

For second order reactions, equimolar concentrations of u-PA and PAI-1 were mixed directly in microtiter plate wells and preincubated at room temperature for periods of time varying from 0 to 30 minutes. Following preincubation the mixtures were quenched with an excess of neutralizing anti-PAI-1 antibody (generously provided by Dr. David Loskutoff), and residual enzymatic activity was measured using a standard, indirect chromogenic assay. These indirect, chromogenic assays were compared to control reactions containing no PAI-1 or to which PAI-1 was added after preincubation, addition of anti-PAI-1 antibody, plasminogen, and Spec PL to the reaction mixture. Data were analyzed by plotting the reciprocal of the residual enzyme concentration versus the time of preincubation.

To test the prediction, based on an analysis of the cleavage of peptide substrates, that the P3 residues can mediate the ability of an inhibitor to discriminate between t-PA and u-PA, site-specific mutagenesis was performed on PAI-1, the primary physiological inhibitor of both t-PA and u-PA.

Three variants of PAI-1 were produced and characterized. The first was a variant in which the P3 serine (Ser 344) was converted to an arginine residue. The second was a variant in which the P4 valine (Val 343) was replaced by a glutamine residue. The third variant was a double mutant in which both of the substitutions were made.

Kinetic analysis of the inhibition of both t-PA and u-PA by these variants of PAI-1 was consistent with the tests based on the peptide substrates. The second-order rate constants for inhibition of t-PA and u-PA by wild type PAI-1 were 1.6×10⁶ M⁻¹s⁻¹ and 1.9×10⁷ M⁻¹s⁻¹, respectively. Thus, wild-type PAI-1 shows about 11.9-fold specificity toward u-PA.

In contrast, the second-order rate constants for inhibition of t-PA and u-PA by the P3 arginine mutant PAI-1 were, respectively, 1.4×10⁶ M⁻¹s⁻¹ and 1.0×10⁵ M⁻¹s⁻¹, an approximately 170-fold reversal in specificity toward t-PA. This large change in specificity was achieved without sacrificing activity toward the target enzyme. The P3 arginine mutation reduced activity of PAI-1 toward u-PA by a factor of about 190 without significantly affecting reactivity toward t-PA.

An individual mutation of the P4 valine to a glutamine had no effect on the rate of inhibition of either t-PA or u-PA. As suggested by the predominance in the subtraction library of substrates containing both large P3 and large P4 residues, the P4 glutamine mutation did increase the t-PA selectivity of the P3 arginine variant of PAI-1. The second order rate constants for the inhibition of t-PA and u-PA by this double mutant PAI-1 were, respectively, 1.4×10⁶ M⁻¹s⁻¹ and 2.9×10⁴ M⁻¹s⁻¹. While maintaining full activity toward t-PA, the double mutant showed an approximately 600-fold enhanced t-PA/u-PA selectivity compared to wild-type PAI-1 and about a 3.5-fold greater t-PA selectivity than the P3 arginine variant of PAI-1. The absolute t-PA/u-PA selectivity of wild-type PAI-1, the P3 arginine single mutant and the P3 Arg, P4 Gln double mutant was 0.08, 14, and 48, respectively.

TABLE 5

Second order rate constants for inhibition of t-PA or u-PA
by wild type PAI-1 and variants of PAI-1

| | | | | | |
|---|---|---|---|---|---|
| | | Primary | Rate | Rate | |
| | | Sequence of | constant | constant | |
| | SEQ ID | reactive center | toward t-PA | toward u-PA | t-PA/u-PA |
| Inhibitor | NO: | loop (P4-P2′) | M⁻¹s⁻¹ | M⁻¹s⁻¹ | Selectivity |

| | | | | | |
|---|---|---|---|---|---|
| Wild type PAI-1 | 149 | VSAR↓MA | 1.6 × 10⁶ | 1.9 × 10⁷ | 0.08 |
| PAI-1/P3R | 150 | VRAR↓MA | 1.4 × 10⁶ | 1.0 × 10⁵ | 14 |
| PAI-1/P4Q | 151 | QSAR↓MA | 1.6 × 10⁶ | 1.9 × 10⁷ | 0.08 |
| PAI-1/P4Q, P3R | 152 | QRAR↓MA | 1.4 × 10⁶ | 2.9 × 10⁴ | 48 |

### EXAMPLE 3

### Preparation of Specific Inhibitors of Urokinase

Substrate phage display alone, without subtractive substrate screening, was to identify peptides that are cleaved 840-5300 times more efficiently by u-PA than peptides containing the wild type physiological target sequence of the enzyme. In addition, the peptide substrates selected were cleaved as much as 120 times more efficiently by u-PA than by t-PA.

In general, with the exception of the screening protocol, procedures followed were those used in Example 1. Digestion of the phage was performed using enzyme concentrations varying from 2-10 μg/ml and incubation times varying from 0.5-10 hours. Phage precipitation and dot blot analysis were performed as described in Example 1. Individual phage stocks were prepared and digested with no enzyme, t-PA, u-PA, or u-PA in the presence of 1 mM amiloride, a specific inhibitor of u-PA′ for periods of time varying from 15 minutes to 10 hours. Individual reaction mixtures were spotted onto a nitrocellulose filter using a dot blotter apparatus (BioRad). The filter was probed with MAb 3E-7 and developed using the Amersham Western ECL kit. Loss of positive staining indicates loss of antibody epitopes from the phage due to proteolytic cleavage of the randomized hexamer region.

Kinetic data were obtained by incubating various concentrations of peptide with a constant enzyme concentration to achieve between 5 and 20% cleavage of the peptide in each reaction as described in Example 1.

A polyvalent fd phage library that displayed random hexapeptide sequences and contained 2×10⁸ independent recombinants was prepared. Each member of this library displayed an N-terminal extension from phage coat protein III (pIII) that contained a randomized region of six amino acids, a six residue linker sequence (SSGGSG), and the epitopes for mAb 179 and mAb 3-E7. Because u-PA did not digest the plII sequence, the antibody epitopes, or the flexible linker sequence, the loss of antibody epitopes from the phage surface upon incubation with u-PA required cleavage of the randomized peptide insert. Incubation of the library with u-PA, followed by removal of phage retaining the antibody epitopes, therefore, accomplished a large enrichment of phage clones whose random hexamer sequence could be cleaved by u-PA.

Following five rounds of selection to enrich and amplify phage which display sequences that are readily cleaved by u-PA, 100 phage clones were identified as u-PA substrates. DNA sequencing of these clones revealed the presence of 91 distinct hexamer sequences among the selected phage (Table 6, below). As expected from the trypsin-like primary specificity of u-PA, each hexamer contained at least one basic residue, and 89 of the 91 hexamer sequences contained at least one arginine residue. 35 of the 91 substrate phage contained a single basic residue, and in 33 of these 35 cases the single basic residue was an arginine. An additional 22 phage contained two basic residues but only a single arginine. Alignment and analysis of these hexamer sequences suggested that the consensus sequence for optimal subsite occupancy for substrates of u-PA, from P3-P2′, was SGR(S>R,K,A)X, where “X” represents a variety of amino acid residues but is most often alanine, glycine, serine, valine, or arginine.

Analysis of these data was complicated by the fact that approximately 72% of the selected substrate phage contained an arginine in the first position of the randomized hexamer and therefore utilized the amino terminal flanking residues, Ser-Gly, to occupy the P3 and P2 subsites. While these results left no doubt that the P3-PT SGR sequence created by the fusion was a very favorable recognition site for u-PA, this use of flanking residues necessitated a particularly careful examination of the P3 and P2 preferences of u-PA.

Two changes were made in the experimental protocol to examine the P3 and P2 preferences of u-PA. First, an unusually large collection of substrate phage (91) were isolated to assure that a reasonable number of these (23) would not utilize the flanking Ser-Gly to fill the P3 and P2 subsites. This allowed a meaningful comparison of the consensus sequence derived from the entire library with that derived from the non-fusion phage and the demonstration of good agreement between the two consensus sequences. Second, dot blot analysis was performed as described in Example 1 on all 100 substrate phage using a wide variety of stringencies of digestion by u-PA. Although this semi-quantitative assay cannot provide kinetic constants, it can provide an accurate rank ordering of the lability of the substrate phage clones.

Under the most stringent conditions examined, 11 of the 100 substrate phage, containing 8 distinct randomized hexamer sequences, proved to be particularly labile u-PA substrates. The sequences were the same as those listed in Table 3, above. All 8 of the most labile substrate phage contained the P3-P1 SGR motif, demonstrating that this sequence is, in fact, a more labile u-PA site than related, selected sequences present in the library such as SSR, TAR, TSR, TTR, etc. This dot blot analysis also yielded additional information regarding the preferences of u-PA for the unprimed subsites. While analysis of the entire substrate phage library failed to reveal a clear consensus at P1′ and P2′ the most labile substrate phage displayed an obvious preference at both of these positions. Five of the eight most labile phage contained a serine residue at P1′, and seven of these eight phage contained an alanine residue at P2′. These observations strongly suggest that the primary sequence SGRSA, from P3-P2′, represents optimal subsite occupancy for substrates of u-PA.

TABLE 6

Amino acid sequences of the hexapeptide in 89
isolated substrate phage clones.

| | | |
|---|---|---|
| Clone | Amino acid | |
| Number | Sequence | SEQ ID NO: |

| | | |
|---|---|---|
| 1 | S G R A R Q | 153 |
| 2 | S K S G R S(L) | 154 |
| 3 | S S R N A D | 155 |
| 4 | T A R L R G | 156 |
| 5 | T A R S D N | 157 |
| 6 | T S R M G T | 158 |
| 7 | T S R Q A Q | 159 |
| 8 | T T R R N K | 160 |
| 9 | T T S R R S | 161 |
| 10 | W S G R S G | 162 |
| 11 | A I K R S A | 163 |
| 12 | (G)G R R G N R | 164 |
| 13 | (G)G R S V N N | 165 |
| 14 | H T R R M K | 166 |
| 15 | I S T A R M(L) | 167 |
| 16 | (S G)K A A D V T | 168 |
| 17 | K K R T N D | 169 |
| 18 | K M S A R I(L) | 170 |
| 19 | (G)K R R D V A | 171 |
| 20 | (G)K R V S K N | 172 |
| 21 | (S G)K S A D A A | 173 |
| 22 | (S G)R A A A M | 174 |
| 23 | (S G)R A G N I R | 175 |
| 24 | (S G)R A H R D N | 176 |
| 25 | (S G)R A R D D R | 177 |
| 26 | (S G)R A R H M | 178 |
| 27 | (S G)R A R S P R | 179 |
| 28 | (S G)R A V G H Q | 180 |
| 29 | (S G)R A V V D S | 181 |
| 30 | (S G)R G G K G P | 182 |
| 31 | (S G)R G R S A V | 183 |
| 32 | (S G)R G V D M N | 184 |
| 33 | (S G)R G V K M H | 185 |
| 34 | (S G)R H R S D I | 186 |
| 35 | (S G)R K G Q G G | 187 |
| 36 | (S G)R K L H M N | 188 |
| 37 | (S G)R K M D M G | 189 |
| 38 | (S G)R K M D R S | 190 |
| 39 | (S G)R K M R M G | 191 |
| 40 | (S G)R K N Q R V | 192 |
| 41 | (S G)R K Q R D S | 193 |
| 42 | (S G)R K R V G A | 194 |
| 43 | (S G)R K S K V V | 195 |
| 44 | (S G)R K S T S S | 196 |
| 45 | (S G)R K V G S L | 197 |
| 46 | (S G)R K A S L S | 37 |
| 47 | (S G)R K V P G S | 198 |
| 48 | (S G)R K W I S G | 199 |
| 49 | (S G)R L A T K A | 200 |
| 50 | (S G)R M R K N D | 201 |
| 51 | (S G)R N A Q V R | 34 |
| 52 | (S G)R N A V E P | 202 |
| 53 | (S G)R N D R L N | 203 |
| 54 | (S G)R N G K S R | 204 |
| 55 | (S G)R N M P L L | 205 |
| 56 | (S G)R N T G S H | 206 |
| 57 | (S G)R R M T M G | 207 |
| 58 | (S G)R R R L N M | 208 |
| 59 | (S G)R R T L D F | 209 |
| 60 | (S G)R R A V S N | 38 |
| 61 | (S G)R S A K V D | 36 |
| 62 | (S G)R S A N A I | 33 |
| 63 | (S G)R S A T R D | 35 |
| 64 | (S G)R S A V V K | 39 |
| 65 | (S G)R S D Q F L | 210 |
| 66 | (S G)R S D N P N | 211 |
| 67 | (S G)R S E R S L | 212 |
| 68 | (S G)R S G D P G | 213 |
| 69 | (S G)R S G N T T | 214 |
| 70 | (S G)R S G N M G | 215 |
| 71 | (S G)R S N G V G | 216 |
| 72 | (S G)R S P D G M | 217 |
| 73 | (S G)R S R R L P | 218 |
| 74 | (S G)R S R V T S | 219 |
| 75 | (S G)R S S H S S | 220 |
| 76 | (S G)R S S Q A A | 221 |
| 77 | (S G)R S S S S H | 40 |
| 78 | (S G)R S S S T V | 222 |
| 79 | (S G)R S T D L G | 223 |
| 80 | (S G)R S T N V E | 224 |
| 81 | (S G)R S T R H K | 225 |
| 82 | (S G)R S Y T N S | 226 |
| 83 | (S G)R T S P S T | 227 |
| 84 | (S G)R T S V N L | 228 |
| 85 | S K R A S I | 229 |
| 86 | S Q T C V R(L V) | 230 |
| 87 | T E R R V R(L V) | 231 |
| 88 | T Q R S T G | 232 |
| 89 | T R R D R I | 233 |
| 90 | V A R M Y K | 234 |
| 91 | V S R R N M | 235 |

Kinetic Analysis of the Cleavage of Peptides Containing Sequences Present in Selected Substrate Phage.

Four peptides containing amino acid sequences present in the randomized hexamer region of the most labile phage were chosen for detailed kinetic analysis (Table 7) and compared to hydrolysis of a control peptide (I) containing the P3-P4′ sequence of plasminogen, a series of residues which fall within a disulfide-linked loop in the native protein. All four of the selected peptides were substantially improved substrates for u-PA, by factors of 840-5300, compared with the control, plasminogen peptide (Table 7). These increases in catalytic efficiency were mediated primarily by increases in k_{cat}, suggesting that optimized subsite interactions served to lower the energy of the transition state rather than the ground state. For example, compared with that of control peptide (I), the Kₘ for cleavage of the most labile, selected peptide (II) was reduced by a factor of 5.6. However, the k_{cat} was increased by a factor of more than 940. In addition, peptide substrates that interacted optimally with the primary subsites of u-PA were selective for cleavage by u-PA relative to t-PA. The four selected peptides (II-V), for example, were cleaved 16-89 times more efficiently by u-PA than by t-PA, and improvements in both Kₘ and k_{cat} contributed to the preferential hydrolysis by u-PA.

TABLE 7

Comparison of k_{cat}, and Kₘ1, and k_{cat}/Kₘ for the hydrolysis by t-PA or u-PA of peptides
selected for preferential cleavage by u-PA

| | |
|---|---|
| u-PA | t-PA |

| | | | | | | | | |
|---|---|---|---|---|---|---|---|---|
| Substrate | SEQ ID | k_{cat} | Kₘ | k_{cat}/Kₘ | k_{cat} | Kₘ | k_{cat}/Kₘ | u-PA:t-PA |
| ¹(Pn,.P3,P2,P1,↓P1′,P2′,P3′..Pn) | NO: | s⁻¹ | (μM) | (M⁻¹s⁻¹) | s⁻¹ | (μM) | (M⁻¹s⁻¹) | Selectivity |

Native cleavage sequence from Plasminogen

| | | | | | | | | | |
|---|---|---|---|---|---|---|---|---|---|
| (I) | KKSPGR↓WGGSVAH | 1 | 0.003 | 3400 | 0.88 | 0.0043 | 15000 | 0.29 | 3.0 |

u-PA selective peptides

| | | | | | | | | | |
|---|---|---|---|---|---|---|---|---|---|
| (II) | LGGSGR↓SANAILE | 11 | 2.83 | 603 | 4700 | 0.305 | 4080 | 75 | 63 |
| (III) | LGGSGR↓NAQVRLE | 12 | 3.69 | 1160 | 3200 | 0.255 | 7000 | 36 | 89 |
| (IV) | LGGSGR↓SATRDLE | 13 | 0.54 | 733 | 740 | 0.068 | 1500 | 45 | 16 |
| (V) | LGGSGR↓KASLSLE | 14 | 1.14 | 1130 | 1010 | 0.168 | 5100 | 33 | 31 |

Minimized, u-PA selective peptides

| | | | | | | | | | |
|---|---|---|---|---|---|---|---|---|---|
| (VI) | SGR↓S | 15 | 2.3 | 2100 | 1100 | 5.0 | 15000 | 330 | 3.3 |
| (VII) | SGR↓SA | 16 | 3.7 | 3100 | 1200 | 2.4 | 40000 | 60 | 20 |
| (VIII) | SGK↓S | 17 | 1.22 | 7900 | 154 | 0.19 | 28000 | 6.8 | 23 |
| (IX) | GSGK↓S | 18 | 0.82 | 4250 | 193 | 0.07 | 44000 | 1.6 | 121 |

Variants of u-PA selective peplides

| | | | | | | | | | |
|---|---|---|---|---|---|---|---|---|---|
| (X) | LGGYGR↓SANAILE | 236 | 0.7 | 2200 | 318 | 3.29 | 1850 | 1800 | 0.018 |
| (XI) | LGGRGR↓SANAILE | 237 | 0.08 | 1200 | 67 | 0.85 | 2400 | 350 | 0.019 |
| (XII) | LGQRGR↓SANAILE | 238 | 0.068 | 1500 | 45 | 2.55 | 3000 | 850 | 0.005 |

¹Positional nomenclature of subsite residues. Arrows denote the position of peptide bond hydrolysis. The peptide bond is cleaved between P1 and P1′. The error in these determinations was 4-22%.

Minimization of the Selective Peptide Substrates.

The kinetic analysis described above was performed using substrate peptides that were 14 amino acids in length. To confirm that the specificity observed was inherent in the selected hexapeptide sequences, the kinetics of cleavage of short peptides containing only sequences found within selected hexapeptide sequences was examined. Pentapeptide VII, for example, was cleaved by u-PA with a catalytic efficiency of 1200 M⁻¹s⁻¹ and exhibited a u-PA/t-PA selectivity of 20. The behavior of pentamer VII in these assays, therefore, was very similar to that of peptide IV, a 14-mer that contains the same P3-P2′ sequence as the pentamer. These observations indicate that appropriate occupancy of the P3-P2′ subsites alone can create selective substrates for u-PA.

Differences at position 190 (chymotrypsin numbering system) between u-PA and t-PA suggest that u-PA may exhibit decreased discrimination between arginine and lysine at the P1 position of a substrate compared with t-PA. Consistent with this hypothesis and by contrast to the selected t-PA substrate library, the u-PA library did include members that contained a P1 lysine. This observation suggested that the u-PA/t-PA selectivity of a peptide substrate should be enhanced by placement of lysine in the P1 position although this increased selectivity was likely to be accompanied by decreased reactivity toward u-PA. To test this hypothesis we analyzed hydrolysis of a variant of u-PA selective peptide (VI) that contained a P1 lysine (peptide VIII). The P1 lysine mutation decreased the catalytic efficiency for cleavage of this peptide by a factor of 49 for t-PA and by a factor of 7 for u-PA. As predicted, then, the P1 lysine mutation did enhance the u-PA/t-PA selectivity of the peptide substrate by a factor of approximately 7. It is not surprising, therefore, that the most selective u-PA substrate, peptide IX which is cleaved approximately 121 times more efficiently by u-PA than by t-PA, is derived from the randomized hexamer region of a substrate phage that contained a P1 lysine.

Importance of P3 and P4 for Discrimination Between u-PA and t-PA.

Recent investigations that explored optimal subsite occupancy for substrates of t-PA suggested that the P3 residue was the primary determinant of the ability of a substrate to discriminate between t-PA and u-PA and that this selectivity could be enhanced modestly by appropriate occupancy of P4. These suggestions were based on evidence obtained from a statistical analysis of phage selected using a substrate subtraction protocol rather than by a kinetic analysis of peptide substrates. Consequently, to test these hypotheses, we synthesized variants of the most labile u-PA selective substrate (peptide II) that contained mutations in the P3 and/or P4 positions and analyzed the hydrolysis of these peptides by u-PA and t-PA. In peptide X the P3 Serine of peptide II was replaced by a tyrosine, and in peptide XI the P3 serine was replaced by arginine. As expected, these mutations substantially decreased the u-PA/t-PA selectivity of the peptide by factors of 330 or 360, respectively, and actually converted the peptide into a t-PA selective substrate. Moreover, mutation of both the P3 serine and P4 glycine of the most labile u-PA substrate to arginine and glutamine, respectively (peptide XII), decreased the u-PA/t-PA selectivity by a factor of 1200. These data confirm the proposed status of the P3 and P4 residues as specificity determinants for substrates of t-PA and u-PA and suggest a particularly prominent role of the P3 residue in this capacity.

### EXAMPLE 4

### Design and Characterization of a Variant of PAI-1 That is Selective for u-PA

Analysis of the selected peptide substrates identified in Example 3 indicated that the primary sequence SGRSA, from positions P3 to P2′, represented an optimal subsite occupancy for substrates of u-PA. This information was to design a variant of plasminogen activator inhibitor type 1 (PAI-1), the primary physiological inhibitor of both u-PA and t-PA, that inhibited u-PA approximately 70 times more rapidly than it inhibited t-PA.

Specific inhibitors of u-PA were designed using the procedure described in Example 2 by making variants of the PAI-1. Oligonucleotide directed, site specific mutagenesis was used as described in Example 2 to construct a variant of PAI-1 that contained the primary sequence found in the peptide substrate that was most selective for u-PA, GSGKS, from the P4-P1′ position of the reactive center loop. The mutagenic oligonucleotide had the sequence 5′-CCACAGCTGTCATAGGCAGCGGCAAAAGCGCCCCCGAGGAGATC-3′.

Following mutagenesis, single-stranded DNA corresponding to the entire 300-bp SalI-BamHI fragment was fully sequenced to ensure the presence of the desired mutations and the absence of any additional mutations The 300-bp SalI-BamHI dsDNA fragment from the mutated, replicative form DNA was used to replace the corresponding fragment in pPAIST7HS to yield a full-length cDNA encoding PAI-1 /UK1, which contained the amino acid sequence GSGKSA from the P4 to P2′ positions of the reactive center loop.

Kinetic analysis indicated that the PAI-1 variant inhibited u-PA approximately 70 times more rapidly than it inhibited t-PA with second order rate constants for inhibition of u-PA and t-PA of 6.2×10⁶ M⁻¹s⁻¹ and 9×10⁴ M⁻¹s⁻¹, respectively. By contrast, wild type PAI-1 inhibits u-PA and t-PA with second order rate constants of 1.9×10⁷ M⁻¹s⁻¹ Z and 1.8×10⁶ M⁻¹s⁻¹ respectively. As anticipated, therefore, the mutated serpin possessed a u-PA/t-PA selectivity that was approximately 7-fold greater than that of wild type PAI-1. Moreover, the 70-fold selectivity of the PAI-1 variant is consistent with the value of 120 observed for hydrolysis of the corresponding peptide substrate by the two enzymes (Tables 7 and 8).

TABLE 8

Second order rate constants for inhibition of t-PA or u-PA
by wild type PAI-1 and variant PAI-1/UK1

| | | | | | |
|---|---|---|---|---|---|
| | | Primary | Rate | Rate | |
| | | Sequence of | constant | constant | |
| | SEQ ID | reactive center | toward u-PA | toward t-PA | t-PA/u-PA |
| Inhibitor | NO: | loop (P4-P2′) | M⁻¹s⁻¹ | M⁻¹s⁻¹ | Selectivity |

| | | | | | |
|---|---|---|---|---|---|
| Wild type PAI-1 | 149 | VSAR↓MA | 1.9 × 10⁷ | 1.8 × 10⁶ | 11 |
| PAI-1/UK1 | 239 | GSGK↓SA | 6.2 × 10⁶ | 9.0 × 10⁴ | 69 |

Other embodiments of the present invention will be apparent to those skilled in the arts of protein engineering or rational drug design.

We claim:

1. A substrate subtraction library composition comprising a collection of different peptides selected based on a first modification by a first enzyme and selected based on a second modification by a second enzyme, wherein the collection of peptides are substrates for the first enzyme and have a substantially lower activity as substrate for the second enzyme.

2. The composition of claim 1, wherein the peptides have a selectivity for the first enzyme over the second enzyme of at least 10 fold.

3. The composition of claim 1, wherein the peptides have a selectivity for the first enzyme over the second enzyme of at least 50 fold.

4. The composition of claim 1, wherein the peptides have a selectivity for the first enzyme over the second enzyme of at least 1,000 fold.

5. The composition of claim 1, wherein the peptides have a selectivity for the first enzyme over the second enzyme of 10 to 1,000 fold.

6. The composition of claim 1, wherein the peptides have a selectivity for the first enzyme over the second enzyme of more than 9000 fold.

7. The composition of claim 1, wherein the peptides in the library have a k_{cat}/Kₘ ratio of less than 500 M⁻¹s⁻¹ as a substrate for the second enzyme.

8. The composition of claim 1, wherein the peptides in the library have a k_{cat}/Kₘ ratio of less than 100 M⁻¹s⁻¹ as a substrate for the second enzyme.

9. The composition of claim 1, wherein the first enzyme and the second enzyme are in the same enzymatic class.

10. The composition of claim 1, wherein the first enzyme and the second enzyme are proteases.

11. The composition of claim 1, wherein the first enzyme and the second enzyme are serine proteases.

12. The composition of claim 1, wherein the first enzyme and the second enzyme are in the chymotrypsin family of serine proteases.

13. The composition of claim 1, wherein the first enzyme comprises tissue plasminogen activator (t-PA) and the second enzyme comprises urokinase-type plasminogen activator (u-PA).

14. The composition of claim 1, wherein the first enzyme comprises urokinase-type plasminogen activator (u-PA) and the second enzyme comprises tissue plasminogen activator (t-PA).

15. The composition of claim 1, wherein the first enzyme and the second enzyme are kinases.

16. The composition of claim 1, wherein the first enzyme and the second enzyme are phosphatases.

17. A method for producing a substrate subtraction library of peptides wherein each peptide is a substrate for a first enzyme but is not a substrate for a second enzyme, comprising the steps of:
a) providing a combinatorial library comprising components that display different peptides;
b) contacting the combinatorial library with a first enzyme to permit the first enzyme to modify peptides in the library to form a first modified peptide component portion and a first unmodified peptide component portion of the library;
c) separating the first modified portion of the library from the first unmodified portion of the library;
d) contacting the first modified portion of the library with a second enzyme to permit the second enzyme to modify peptides in the first modified portion of the library to form a second modified peptide component portion and a second unmodified peptide component portion of the library;
e) separating the second modified portion of the library from the second unmodified portion of the library; and
f) retaining the second unmodified portion to form a library of peptides that are each a substrate for the first enzyme but not a substrate for the second enzyme.

18. The method of claim 17, wherein the first enzyme and the second enzyme are proteases.

19. The method of claim 17, wherein the first enzyme comprises tissue plasminogen activator (t-PA) and the second enzyme comprises urokinase-type plasminogen activator (u-PA).

20. The method of claim 17, wherein the first enzyme comprises urokinase-type plasminogen activator (u-PA) and the second enzyme comprises tissue plasminogen activator (t-PA).

21. The method of claim 17, wherein the first enzyme and the second enzyme are kinases.

22. The method of claim 17, wherein the first enzyme and the second enzyme are phosphatases.

23. The method of claim 17, wherein the combinatorial library is a bacteriophage display library.

24. A method for producing a substrate subtraction library of peptides wherein each peptide is a substrate for a second enzyme but is not a substrate for a first enzyme, comprising the steps of:
a) providing a combinatorial library comprising components that display different peptides;
b) contacting the combinatorial library with a first enzyme to permit the first enzyme to modify peptides in the library to form a first modified peptide component portion and a first unmodified peptide component portion of the library;
c) separating the first modified portion of the library from the first unmodified portion of the library;
d) contacting the first unmodified portion of the library with a second enzyme to permit the second enzyme to modify peptides to form a second modified peptide component portion and a second unmodified peptide component portion of the library; and
e) separating the second modified portion of the library from the second unmodified portion of the library, and retaining the second modified portion to form a library of peptides that are each a substrate for the second enzyme but not a substrate for the first enzyme.

25. The method of claim 24, wherein the first enzyme and the second enzyme are proteases.

26. The method of claim 24, wherein the first enzyme comprises tissue plasminogen activator (t-PA) and the second enzyme comprises urokinase-type plasminogen activator (u-PA).

27. The method of claim 24, wherein the first enzyme comprises urokinase-type plasminogen activator (u-PA) and the second enzyme comprises tissue plasminogen activator (t-PA).

28. The method of claim 24, wherein the first enzyme and the second enzyme are kinases.

29. The method of claim 24, wherein the first enzyme and the second enzyme are phosphatases.

30. The method of claim 24, wherein the combinatorial library is a bacteriophage display library.

31. A composition comprising a substrate subtraction library comprising a collection of peptides that are selective substrates of a first enzyme over a second enzyme; wherein the collection of peptides are selected from a combinatorial peptide library by substrate subtraction screening based on a first modification by the first enzyme and by substrate subtraction screening based on a second modification by the second enzyme; and wherein the first enzyme and the second enzyme are in the same enzymatic class.

32. The composition of claim 31, wherein the peptides have a selectivity for the first enzyme over the second enzyme of at least 10 fold.

33. The composition of claim 31, wherein the peptides have a selectivity for the first enzyme over the second enzyme of at least 50 fold.

34. The composition of claim 31, wherein the peptides have a selectivity for the first enzyme over the second enzyme of at least 1000 fold.

35. The composition of claim 31, wherein the peptides have a selectivity for the first enzyme over the second enzyme of 10 to 1,000 fold.

36. The composition of claim 31, wherein the peptides have a selectivity for the first enzyme over the second enzyme of more than 9000 fold.

37. A composition comprising a substrate subtraction library comprising a collection of peptides that are selective substrates of a first protease enzyme over a second protease enzyme; wherein the collection of peptides are selected from a combinatorial peptide library by substrate subtraction screening based on cleavage by the first protease enzyme and by substrate subtraction screening based on cleavage by the second protease enzyme.

38. The composition of claim 37, wherein the peptides have a selectivity for the first protease enzyme over the second protease enzyme of at least 10 fold.

39. The composition of claim 37, wherein the peptides have a selectivity for the first protease enzyme over the second protease enzyme of at least 50 fold.

40. The composition of claim 37, wherein the peptides have a selectivity for the first protease enzyme over the second protease enzyme of at least 1000 fold.

41. The composition of claim 37, wherein the peptides have a selectivity for the first enzyme over the second enzyme of 10 to 1,000 fold.

42. The composition of claim 37, wherein the peptides have a selectivity for the first enzyme over the second enzyme of more than 9000 fold.

43. A composition comprising a substrate subtraction library comprising a collection of peptides that are selective substrates of a first kinase enzyme over a second kinase enzyme; wherein the collection of peptides are selected from a combinatorial peptide library by substrate subtraction screening based on activity of the first kinase enzyme and by substrate subtraction screening based on activity of the second kinase enzyme.

44. The composition of claim 43, wherein the peptides have a selectivity for the first kinase enzyme over the second kinase enzyme of at least 10 fold.

45. The composition of claim 43, wherein the peptides have a selectivity for the first kinase enzyme over the second kinase enzyme of at least 50 fold.

46. The composition of claim 43, wherein the peptides have a selectivity for the first kinase enzyme over the second kinase enzyme of at least 1000 fold.

47. A composition comprising a substrate subtraction library comprising a collection of peptides that are selective substrates of a first phosphatase enzyme over a second phosphatase enzyme; wherein the collection of peptides are selected from a combinatorial peptide library by substrate subtraction screening based on activity of the first phosphatase enzyme and by substrate subtraction screening based on activity of the second phosphatase enzyme.

48. The composition of claim 47, wherein the peptides have a selectivity for the first phosphatase enzyme over the second phosphatase enzyme of at least 10 fold.

49. The composition of claim 47, wherein the peptides have a selectivity for the first phosphatase enzyme over the second phosphatase enzyme of at least 50 fold.

50. The composition of claim 47, wherein the peptides have a selectivity for the first phosphatase enzyme over the second phosphatase enzyme of at least 1000 fold.

51. The composition of claim 47, wherein the peptides have a selectivity for the first enzyme over the second enzyme of 10 to 1,000 fold.

52. The composition of claim 47, wherein the peptides have a selectivity for the first enzyme over the second enzyme of more than 9000 fold.

53. A method of providing enzymatically selective peptides, comprising:
a) providing a peptide combinatorial library;
b) using substrate subtraction screening of the combinatorial library to provide a first substrate subtraction library of peptides that are substrates of a first enzyme;
c) using substrate subtraction screening of the first enzyme substrate subtraction library to provide a second substrate subtraction library of peptides lacking substrates of the second enzyme; thereby providing the peptides having the enzymatically selective peptides.

54. The method of claim 53, further comprising identifying an amino acid sequence of one or more of the enzymatically selective peptides.

55. The method of claim 53, wherein the peptides have a selectivity for the first enzyme over the second enzyme of at least 10 fold.

56. The method of claim 53, wherein the peptides have a selectivity for the first enzyme over the second enzyme of at least 50 fold.

57. The method of claim 53, wherein the peptides have a selectivity for the first enzyme over the second enzyme of at least 1000 fold.

58. The method of claim 53, wherein the first enzyme and the second enzyme are proteases.

59. The method of claim 53, wherein the first enzyme and the second enzyme are kinases.

60. The method of claim 53, wherein the first enzyme and the second enzyme are phosphatases.

61. A method of providing enzymatically selective peptides, comprising:
a) providing a peptide combinatorial library;
b) using substrate subtraction screening of the combinatorial library to provide a first substrate subtraction library of peptides that lacking substrates of a first enzyme;
c) using substrate subtraction screening of the first enzyme substrate subtraction library to provide a second substrate subtraction library of peptides that are substrates of the second enzyme; thereby providing the peptides having the enzymatically selective peptides.

62. The method of claim 61, further comprising identifying an amino acid sequence of one or more of the enzymatically selective peptides.

63. The method of claim 61, wherein the peptides have a selectivity for the first enzyme over the second enzyme of at least 10 fold.

64. The method of claim 61, wherein the peptides have a selectivity for the first enzyme over the second enzyme of at least 50 fold.

65. The method of claim 61, wherein the peptides have a selectivity for the first enzyme over the second enzyme of at least 1000 fold.

66. The method of claim 61, wherein the first enzyme and the second enzyme are proteases.

67. The method of claim 61, wherein the first enzyme and the second enzyme are kinases.

68. The method of claim 61, wherein the first enzyme and the second enzyme are phosphatases.


SEQUENCE LISTING (244 sequences)

SEQ ID NO: 1 - protein, 14 aa, Homo sapiens
KKSPGRVVGGSVAH

SEQ ID NO: 2 - protein, 13 aa, Homo sapiens
LGGSGQRGRKALE

SEQ ID NO: 3 - protein, 13 aa, Homo sapiens
LGGSGERARGALE

SEQ ID NO: 4 - protein, 13 aa, Homo sapiens
LGGSGHYGRSGLE

SEQ ID NO: 5 - protein, 4 aa, Homo sapiens
YGRS

SEQ ID NO: 6 - protein, 4 aa, Homo sapiens
RGRK

SEQ ID NO: 7 - protein, 5 aa, Homo sapiens
FRGRK

SEQ ID NO: 8 - protein, 13 aa, Homo sapiens
LGGYGRSANAILE

SEQ ID NO: 9 - protein, 13 aa, Homo sapiens
LGGRGRSANAILE

SEQ ID NO: 10 - protein, 13 aa, Homo sapiens
LGQRGRSANAILE

SEQ ID NO: 11 - protein, 13 aa, Homo sapiens
LGGSGRSANAILE

SEQ ID NO: 12 - protein, 13 aa, Homo sapiens
LGGSGRNAQVRLE

SEQ ID NO: 13 - protein, 13 aa, Homo sapiens
LGGSGRSATRDLE

SEQ ID NO: 14 - protein, 13 aa, Homo sapiens
LGGSGRKASLSLE

SEQ ID NO: 15 - protein, 4 aa, Homo sapiens
SGRS

SEQ ID NO: 16 - protein, 5 aa, Homo sapiens
SGRSA

SEQ ID NO: 17 - protein, 4 aa, Homo sapiens
SGKS

SEQ ID NO: 18 - protein, 5 aa, Homo sapiens
GSGKS

SEQ ID NO: 19 - protein, 6 aa, Homo sapiens
ALRRGD

SEQ ID NO: 20 - protein, 7 aa, Homo sapiens
DYRGRML

SEQ ID NO: 21 - protein, 6 aa, Homo sapiens
ERARGA

SEQ ID NO: 22 - protein, 6 aa, Homo sapiens
ERLRKA

SEQ ID NO: 23 - protein, 6 aa, Homo sapiens
FGRHAA

SEQ ID NO: 24 - protein, 6 aa, Homo sapiens
FLPRTA

SEQ ID NO: 25 - protein, 6 aa, Homo sapiens
FRGRAA

SEQ ID NO: 26 - protein, 6 aa, Homo sapiens
HRMRMG

SEQ ID NO: 27 - protein, 6 aa, Homo sapiens
HYGRSG

SEQ ID NO: 28 - protein, 6 aa, Homo sapiens
IMRRGK

SEQ ID NO: 29 - protein, 7 aa, Homo sapiens
ITYGRRL

SEQ ID NO: 30 - protein, 6 aa, Homo sapiens
KFTRSG

SEQ ID NO: 31 - protein, 6 aa, Homo sapiens
LIPRRA

SEQ ID NO: 32 - protein, 7 aa, Homo sapiens
MTRKRML

SEQ ID NO: 33 - protein, 6 aa, Homo sapiens
NFARMG

SEQ ID NO: 34 - protein, 6 aa, Homo sapiens
NHLRKA

SEQ ID NO: 35 - protein, 6 aa, Homo sapiens
NVGRMG

SEQ ID NO: 36 - protein, 6 aa, Homo sapiens
NVSRRG

SEQ ID NO: 37 - protein, 6 aa, Homo sapiens
PISRRA

SEQ ID NO: 38 - protein, 6 aa, Homo sapiens
PVGRMG

SEQ ID NO: 39 - protein, 6 aa, Homo sapiens
QRGRKA

SEQ ID NO: 40 - protein, 6 aa, Homo sapiens
RLLRSV

SEQ ID NO: 41 - protein, 6 aa, Homo sapiens
SFGRRH

SEQ ID NO: 42 - protein, 7 aa, Homo sapiens
SLRGRSL

SEQ ID NO: 43 - protein, 6 aa, Homo sapiens
TVLRRA

SEQ ID NO: 44 - protein, 6 aa, Homo sapiens
VARRVK

SEQ ID NO: 45 - protein, 6 aa, Homo sapiens
VIARSN

SEQ ID NO: 46 - protein, 6 aa, Homo sapiens
VNTKSG

SEQ ID NO: 47 - protein, 6 aa, Homo sapiens
VRARGA

SEQ ID NO: 48 - protein, 7 aa, Homo sapiens
VRRGRSL

SEQ ID NO: 49 - protein, 6 aa, Homo sapiens
VRRRGA

SEQ ID NO: 50 - protein, 6 aa, Homo sapiens
TRVRAK

SEQ ID NO: 51 - protein, 8 aa, Homo sapiens
SGRSANAI

SEQ ID NO: 52 - protein, 8 aa, Homo sapiens
SGRNAQVR

SEQ ID NO: 53 - protein, 8 aa, Homo sapiens
SGRSATRD

SEQ ID NO: 54 - protein, 8 aa, Homo sapiens
SGRSAKVD

SEQ ID NO: 55 - protein, 8 aa, Homo sapiens
SGRKASLS

SEQ ID NO: 56 - protein, 8 aa, Homo sapiens
SGRRAVSN

SEQ ID NO: 57 - protein, 8 aa, Homo sapiens
SGRSAVVK

SEQ ID NO: 58 - protein, 8 aa, Homo sapiens
SGRSSSSH

SEQ ID NO: 59 - protein, 6 aa, Homo sapiens
AIKRSA

SEQ ID NO: 60 - protein, 6 aa, Homo sapiens
GRRGNR

SEQ ID NO: 61 - protein, 6 aa, Homo sapiens
GRSVNN

SEQ ID NO: 62 - protein, 6 aa, Homo sapiens
HTRRMK

SEQ ID NO: 63 - protein, 6 aa, Homo sapiens
ISTARM

SEQ ID NO: 64 - protein, 6 aa, Homo sapiens
KAADVT

SEQ ID NO: 65 - protein, 6 aa, Homo sapiens
KKRTND

SEQ ID NO: 66 - protein, 6 aa, Homo sapiens
KMSARI

SEQ ID NO: 67 - protein, 6 aa, Homo sapiens
KRRDVA

SEQ ID NO: 68 - protein, 6 aa, Homo sapiens
KRVSKN

SEQ ID NO: 69 - protein, 6 aa, Homo sapiens
KSADAA

SEQ ID NO: 70 - protein, 6 aa, Homo sapiens
RAAAMV

SEQ ID NO: 71 - protein, 6 aa, Homo sapiens
RAGNIR

SEQ ID NO: 72 - protein, 6 aa, Homo sapiens
RAHRDN

SEQ ID NO: 73 - protein, 6 aa, Homo sapiens
RARDDR

SEQ ID NO: 74 - protein, 6 aa, Homo sapiens
RARHMV

SEQ ID NO: 75 - protein, 6 aa, Homo sapiens
RARSPR

SEQ ID NO: 76 - protein, 6 aa, Homo sapiens
RAVGHQ

SEQ ID NO: 77 - protein, 6 aa, Homo sapiens
RAVVDS

SEQ ID NO: 78 - protein, 6 aa, Homo sapiens
RGGKGP

SEQ ID NO: 79 - protein, 6 aa, Homo sapiens
RGRSAV

SEQ ID NO: 80 - protein, 6 aa, Homo sapiens
RGVDMN

SEQ ID NO: 81 - protein, 6 aa, Homo sapiens
RGVKMH

SEQ ID NO: 82 - protein, 6 aa, Homo sapiens
RHRSDI

SEQ ID NO: 83 - protein, 6 aa, Homo sapiens
RKGQGG

SEQ ID NO: 84 - protein, 6 aa, Homo sapiens
RKLHMN

SEQ ID NO: 85 - protein, 6 aa, Homo sapiens
RKMDMG

SEQ ID NO: 86 - protein, 6 aa, Homo sapiens
RKMDRS

SEQ ID NO: 87 - protein, 6 aa, Homo sapiens
RKMRMG

SEQ ID NO: 88 - protein, 6 aa, Homo sapiens
RKNQRV

SEQ ID NO: 89 - protein, 6 aa, Homo sapiens
RKQRDS

SEQ ID NO: 90 - protein, 6 aa, Homo sapiens
RKRVGA

SEQ ID NO: 91 - protein, 6 aa, Homo sapiens
RKSKVV

SEQ ID NO: 92 - protein, 6 aa, Homo sapiens
RKSTSS

SEQ ID NO: 93 - protein, 6 aa, Homo sapiens
RKVGSL

SEQ ID NO: 94 - protein, 6 aa, Homo sapiens
RKVPGS

SEQ ID NO: 95 - protein, 6 aa, Homo sapiens
RKWISG

SEQ ID NO: 96 - protein, 6 aa, Homo sapiens
RLATKA

SEQ ID NO: 97 - protein, 6 aa, Homo sapiens
RMRKND

SEQ ID NO: 98 - protein, 6 aa, Homo sapiens
RNAQVR

SEQ ID NO: 99 - protein, 6 aa, Homo sapiens
RNAVEP

SEQ ID NO: 100 - protein, 6 aa, Homo sapiens
RNDRLN

SEQ ID NO: 101 - protein, 6 aa, Homo sapiens
RNGKSR

SEQ ID NO: 102 - protein, 6 aa, Homo sapiens
RNMPLL

SEQ ID NO: 103 - protein, 6 aa, Homo sapiens
RNTGSH

SEQ ID NO: 104 - protein, 6 aa, Homo sapiens
RRMTMG

SEQ ID NO: 105 - protein, 6 aa, Homo sapiens
RRRLNM

SEQ ID NO: 106 - protein, 6 aa, Homo sapiens
RRTLDF

SEQ ID NO: 107 - protein, 6 aa, Homo sapiens
RSAKVD

SEQ ID NO: 108 - protein, 6 aa, Homo sapiens
RSANAI

SEQ ID NO: 109 - protein, 6 aa, Homo sapiens
RSATRD

SEQ ID NO: 110 - protein, 6 aa, Homo sapiens
RSAVVK

SEQ ID NO: 111 - protein, 6 aa, Homo sapiens
RSDQFL

SEQ ID NO: 112 - protein, 6 aa, Homo sapiens
RSDNPN

SEQ ID NO: 113 - protein, 6 aa, Homo sapiens
RSERSL

SEQ ID NO: 114 - protein, 6 aa, Homo sapiens
RSGDPG

SEQ ID NO: 115 - protein, 6 aa, Homo sapiens
RSGNTT

SEQ ID NO: 116 - protein, 6 aa, Homo sapiens
RSGNMG

SEQ ID NO: 117 - protein, 6 aa, Homo sapiens
RSNGVG

SEQ ID NO: 118 - protein, 6 aa, Homo sapiens
RSPDGM

SEQ ID NO: 119 - protein, 6 aa, Homo sapiens
RSRRLP

SEQ ID NO: 120 - protein, 6 aa, Homo sapiens
RSRVTS

SEQ ID NO: 121 - protein, 6 aa, Homo sapiens
RSSHSS

SEQ ID NO: 122 - protein, 6 aa, Homo sapiens
RSSQAA

SEQ ID NO: 123 - protein, 6 aa, Homo sapiens
RSSSSH

SEQ ID NO: 124 - protein, 6 aa, Homo sapiens
RSSSTV

SEQ ID NO: 125 - protein, 6 aa, Homo sapiens
RSTDLG

SEQ ID NO: 126 - protein, 6 aa, Homo sapiens
RSTNVE

SEQ ID NO: 127 - protein, 6 aa, Homo sapiens
RSTRHK

SEQ ID NO: 128 - protein, 6 aa, Homo sapiens
RSYTNS

SEQ ID NO: 129 - protein, 6 aa, Homo sapiens
RTSPST

SEQ ID NO: 130 - protein, 6 aa, Homo sapiens
RTSVNL

SEQ ID NO: 131 - protein, 6 aa, Homo sapiens
SGRARQ

SEQ ID NO: 132 - protein, 6 aa, Homo sapiens
SKRASI

SEQ ID NO: 133 - protein, 6 aa, Homo sapiens
SKSGRS

SEQ ID NO: 134 - protein, 6 aa, Homo sapiens
SQTCVR

SEQ ID NO: 135 - protein, 6 aa, Homo sapiens
SSRNAD

SEQ ID NO: 136 - protein, 6 aa, Homo sapiens
TARLRG

SEQ ID NO: 137 - protein, 6 aa, Homo sapiens
TARSDN

SEQ ID NO: 138 - protein, 6 aa, Homo sapiens
TERRVR

SEQ ID NO: 139 - protein, 6 aa, Homo sapiens
TQRSTG

SEQ ID NO: 140 - protein, 6 aa, Homo sapiens
TRRDRI

SEQ ID NO: 141 - protein, 6 aa, Homo sapiens
TSRMGT

SEQ ID NO: 142 - protein, 6 aa, Homo sapiens
TSRQAQ

SEQ ID NO: 143 - protein, 6 aa, Homo sapiens
TTRRNK

SEQ ID NO: 144 - protein, 6 aa, Homo sapiens
TTSRRS

SEQ ID NO: 145 - protein, 6 aa, Homo sapiens
VARMYK

SEQ ID NO: 146 - protein, 6 aa, Homo sapiens
VSRRNM

SEQ ID NO: 147 - protein, 6 aa, Homo sapiens
WSGRSG

SEQ ID NO: 148 - protein, 6 aa, Homo sapiens
RIARRA

SEQ ID NO: 149 - protein, 6 aa, Homo sapiens
VSARMA

SEQ ID NO: 150 - protein, 6 aa, Homo sapiens
VRARMA

SEQ ID NO: 151 - protein, 6 aa, Homo sapiens
QSARMA

SEQ ID NO: 152 - protein, 6 aa, Homo sapiens
QRARMA

SEQ ID NO: 153 - protein, 5 aa, Homo sapiens
SGRAR

SEQ ID NO: 154 - protein, 7 aa, Homo sapiens
SKSGRSL

SEQ ID NO: 155 - protein, 6 aa, Homo sapiens
SSRNAD

SEQ ID NO: 156 - protein, 6 aa, Homo sapiens
TARLRG

SEQ ID NO: 157 - protein, 6 aa, Homo sapiens
TARSDN

SEQ ID NO: 158 - protein, 6 aa, Homo sapiens
TSRMGT

SEQ ID NO: 159 - protein, 6 aa, Homo sapiens
TSRQAQ

SEQ ID NO: 160 - protein, 6 aa, Homo sapiens
TTRRNK

SEQ ID NO: 161 - protein, 6 aa, Homo sapiens
TTSRRS

SEQ ID NO: 162 - protein, 6 aa, Homo sapiens
WSGRSG

SEQ ID NO: 163 - protein, 6 aa, Homo sapiens
AIKRSA

SEQ ID NO: 164 - protein, 7 aa, Homo sapiens
GGRRGNR

SEQ ID NO: 165 - protein, 7 aa, Homo sapiens
GGRSVNN

SEQ ID NO: 166 - protein, 6 aa, Homo sapiens
HTRRMK

SEQ ID NO: 167 - protein, 7 aa, Homo sapiens
ISTARML

SEQ ID NO: 168 - protein, 8 aa, Homo sapiens
SGKAADVT

SEQ ID NO: 169 - protein, 6 aa, Homo sapiens
KKRTND

SEQ ID NO: 170 - protein, 7 aa, Homo sapiens
KMSARIL

SEQ ID NO: 171 - protein, 7 aa, Homo sapiens
GKRRDVA

SEQ ID NO: 172 - protein, 7 aa, Homo sapiens
GKRVSKN

SEQ ID NO: 173 - protein, 8 aa, Homo sapiens
SGKSADAA

SEQ ID NO: 174 - protein, 7 aa, Homo sapiens
SGRAAAM

SEQ ID NO: 175 - protein, 8 aa, Homo sapiens
SGRAGNIR

SEQ ID NO: 176 - protein, 8 aa, Homo sapiens
SGRAHRDN

SEQ ID NO: 177 - protein, 8 aa, Homo sapiens
SGRARDDR

SEQ ID NO: 178 - protein, 7 aa, Homo sapiens
SGRARHM

SEQ ID NO: 179 - protein, 8 aa, Homo sapiens
SGRARSPR

SEQ ID NO: 180 - protein, 8 aa, Homo sapiens
SGRAVGHQ

SEQ ID NO: 181 - protein, 8 aa, Homo sapiens
SGRAVVDS

SEQ ID NO: 182 - protein, 8 aa, Homo sapiens
SGRGGKGP

SEQ ID NO: 183 - protein, 8 aa, Homo sapiens
SGRGRSAV

SEQ ID NO: 184 - protein, 8 aa, Homo sapiens
SGRGVDMN

SEQ ID NO: 185 - protein, 8 aa, Homo sapiens
SGRGVKMH

SEQ ID NO: 186 - protein, 8 aa, Homo sapiens
SGRHRSDI

SEQ ID NO: 187 - protein, 8 aa, Homo sapiens
SGRKGQGG

SEQ ID NO: 188 - protein, 8 aa, Homo sapiens
SGRKLHMN

SEQ ID NO: 189 - protein, 8 aa, Homo sapiens
SGRKMDMG

SEQ ID NO: 190 - protein, 8 aa, Homo sapiens
SGRKMDRS

SEQ ID NO: 191 - protein, 8 aa, Homo sapiens
SGRKMRMG

SEQ ID NO: 192 - protein, 8 aa, Homo sapiens
SGRKNQRV

SEQ ID NO: 193 - protein, 8 aa, Homo sapiens
SGRKQRDS

SEQ ID NO: 194 - protein, 8 aa, Homo sapiens
SGRKRVGA

SEQ ID NO: 195 - protein, 8 aa, Homo sapiens
SGRKSKVV

SEQ ID NO: 196 - protein, 8 aa, Homo sapiens
SGRKSTSS

SEQ ID NO: 197 - protein, 8 aa, Homo sapiens
SGRKVGSL

SEQ ID NO: 198 - protein, 8 aa, Homo sapiens
SGRKVPGS

SEQ ID NO: 199 - protein, 8 aa, Homo sapiens
SGRKWISG

SEQ ID NO: 200 - protein, 8 aa, Homo sapiens
SGRLATKA

SEQ ID NO: 201 - protein, 8 aa, Homo sapiens
SGRMRKND

SEQ ID NO: 202 - protein, 8 aa, Homo sapiens
SGRNAVEP

SEQ ID NO: 203 - protein, 8 aa, Homo sapiens
SGRNDRLN

SEQ ID NO: 204 - protein, 8 aa, Homo sapiens
SGRNGKSR

SEQ ID NO: 205 - protein, 8 aa, Homo sapiens
SGRNMPLL

SEQ ID NO: 206 - protein, 8 aa, Homo sapiens
SGRNTGSH

SEQ ID NO: 207 - protein, 8 aa, Homo sapiens
SGRRMTMG

SEQ ID NO: 208 - protein, 8 aa, Homo sapiens
SGRRRLNM

SEQ ID NO: 209 - protein, 8 aa, Homo sapiens
SGRRTLDF

SEQ ID NO: 210 - protein, 8 aa, Homo sapiens
SGRSDQFL

SEQ ID NO: 211 - protein, 8 aa, Homo sapiens
SGRSDNPN

SEQ ID NO: 212 - protein, 8 aa, Homo sapiens
SGRSERSL

SEQ ID NO: 213 - protein, 8 aa, Homo sapiens
SGRSGDPG

SEQ ID NO: 214 - protein, 8 aa, Homo sapiens
SGRSGNTT

SEQ ID NO: 215 - protein, 8 aa, Homo sapiens
SGRSGNMG

SEQ ID NO: 216 - protein, 8 aa, Homo sapiens
SGRSNGVG

SEQ ID NO: 217 - protein, 8 aa, Homo sapiens
SGRSPDGM

SEQ ID NO: 218 - protein, 8 aa, Homo sapiens
SGRSRRLP

SEQ ID NO: 219 - protein, 8 aa, Homo sapiens
SGRSRVTS

SEQ ID NO: 220 - protein, 8 aa, Homo sapiens
SGRSSHSS

SEQ ID NO: 221 - protein, 8 aa, Homo sapiens
SGRSSQAA

SEQ ID NO: 222 - protein, 8 aa, Homo sapiens
SGRSSSTV

SEQ ID NO: 223 - protein, 8 aa, Homo sapiens
SGRSTDLG

SEQ ID NO: 224 - protein, 8 aa, Homo sapiens
SGRSTNVE

SEQ ID NO: 225 - protein, 8 aa, Homo sapiens
SGRSTRHK

SEQ ID NO: 226 - protein, 8 aa, Homo sapiens
SGRSYTNS

SEQ ID NO: 227 - protein, 8 aa, Homo sapiens
SGRTSPST

SEQ ID NO: 228 - protein, 8 aa, Homo sapiens
SGRTSVNL

SEQ ID NO: 229 - protein, 6 aa, Homo sapiens
SKRASI

SEQ ID NO: 230 - protein, 8 aa, Homo sapiens
SQTCVRLV

SEQ ID NO: 231 - protein, 8 aa, Homo sapiens
TERRVRLV

SEQ ID NO: 232 - protein, 6 aa, Homo sapiens
TQRSTG

SEQ ID NO: 233 - protein, 6 aa, Homo sapiens
TRRDRI

SEQ ID NO: 234 - protein, 6 aa, Homo sapiens
VARMYK

SEQ ID NO: 235 - protein, 6 aa, Homo sapiens
VSRRNM

SEQ ID NO: 236 - protein, 13 aa, Homo sapiens
LGGYGRSANAILE

SEQ ID NO: 237 - protein, 13 aa, Homo sapiens
LGGRGRSANAILE

SEQ ID NO: 238 - protein, 13 aa, Homo sapiens
LGQRGRSANAILE

SEQ ID NO: 239 - protein, 6 aa, Homo sapiens
GSGKSA

SEQ ID NO: 240 - DNA, 46 bp, Artificial Sequence
  Description of Artificial Sequence oligonucleotide
tcgagcggtggatccggtactggtcgtactggtcatgctctggtac

SEQ ID NO: 241 - DNA, 38 bp, Artificial Sequence
  Description of Artificial Sequence oligonucleotide
cgccacctaggccaggaccagcacaacaaccacgagac

SEQ ID NO: 242 - protein, 6 aa, Homo sapiens
SSGGSG

SEQ ID NO: 243 - protein, 6 aa, Homo sapiens
QRGRSA

SEQ ID NO: 244 - DNA, 44 bp, Artificial Sequence
  Description of Artificial Sequence oligonucleotide
ccacagctgtcataggcagcggcaaaagcgcccccgaggagatc